(19) United States
(12) Patent Application Publication
(10) Pub. No.: US 2015/0125432 A1
(43) Pub. Date: May 7, 2015

(54) Human Persistent Fetal Vasculature Neural Progenitors for Transplantation in the Inner Retina

(71) Applicant: The Schepens Eye Research Institute, Inc., Boston, MA (US)

(72) Inventors: Kameran Lashkari, Boston, MA (US); Jie Ma, Malden, MA (US)

(21) Appl. No.: 14/399,827

(22) PCT Filed: May 10, 2013
(86) PCT No.: PCT/US2013/040556

§ 371 Date: Nov. 7, 2014

Related U.S. Application Data
(60) Provisional application No. 61/645,318, filed on May 10, 2012.

(51) Int. Cl. A61K 35/54; A61K 9/00

(52) U.S. Cl. CPC A61K 35/54; A61K 9/0048; A61K 48/00
USPC 424/93.21; 435/325; 435/366; 435/375

(57) ABSTRACT

The invention provides the human persistent fetal vasculature neural progenitor cells for transplantation or other uses such as drug discovery. For example, a cell-based method of therapy is carried out by providing a purified population of human persistent fetal vasculature neural progenitor cells and transplanting the cells into an ocular tissue of a recipient subject.

### RELATED APPLICATION INFORMATION

This application claims priority to U.S. provisional application Ser. No. 61/645,318, filed May 10, 2012, the contents of which are incorporated herein by reference.

### FIELD OF THE INVENTION

The field of the invention relates to cell therapy.

### BACKGROUND OF THE INVENTION

Inner retinal degenerative diseases, such as selective and progressive loss of retinal ganglion cells (RGCs), pose a major threat to vision. Currently there is no effective cure or treatment to reverse the loss of RGCs. Transplantation of stem or progenitor cells may have great therapeutic potential for treatment of neurodegenerative diseases in general by providing therapeutic benefits through both neuroprotective and cell replacement mechanisms and some regenerative potential of cell transplantation has already been shown for the outer retina. However, it has proven difficult to achieve similar success for replacement of RGCs, as they adopt highly specialized properties and form numerous synaptic connections with other neurons. Due to the inhibitory environment present in the adult neural retina and lineage restriction of engrafted cells, limited integration and RGC-specific differentiation of engrafted cells has been found in the inner retina when cells have been intravitreally transplanted into the uninjured eye.

### SUMMARY OF THE INVENTION

The invention provides the use of human persistent fetal vasculature neural progenitor cells for transplantation. These purified cells integrate into the retinal ganglion cell layer of the eye after transplantation into the eye, e.g., into the vitreous of the eye, thereby overcoming problems and/or drawbacks of earlier approaches to treat such degenerative diseases. For example, a cell-based method of therapy is carried out by providing a purified population of human persistent fetal vasculature neural progenitor cells (hNPPFV) and transplanting the cells into an ocular tissue of a recipient subject. The methods are suitable for not only humans but other animals as well, e.g., companion animals such as dogs, cats, and the like as well as livestock or other animals. The cells are obtained from the individual to be treated or a member of the same species. In one example, the cells are transplanted into an inner retina location of an eye. In some cases, the cells have been modified to increase expression of insulin-like growth factor-1 (IGF-1) or insulin-like growth factor-binding protein (IGFBP)-1. For example, the cells to be transplanted have been transfected with a nucleic acid encoding IGF-1.

Thus, the invention encompasses a human persistent fetal vascular tissue cell or cell line comprising a neuronal progenitor marker such as nestin, Pax6, or Ki67. For example, the marker(s) are expressed on the surface or in the cytoplasm of the cell. In some embodiments, the cell further comprises a retinal neuronal marker such as β-III-tubulin or Brn3a. The cells comprise a neural morphological phenotype or express a mature neuronal marker in the presence of a neural phenotype induction factor such as a an environmental cue, e.g., contact with a neural cell differentiation factor. Examples of such phenotype induction factors or neural cell differentiation factors include small molecules such as retinoic acid or neurotrophins or neurotrophic factors. Neurotrophins are a family of proteins that induce the development, survival, and/or function of neurons.

The term neurotrophin may be used as a synonym for neurotrophic factor, but the term neurotrophin is more generally reserved for four structurally related factors: nerve growth factor (NGF), brain-derived neurotrophic factor (BDNF), neurotrophin-3 (NT-3), and neurotrophin-4 (NT-4), with neurotrophic factor additionally referring to the GDNF family of ligands and ciliary neurotrophic factor (CNTF). Exemplary mature neuronal marker include β-III-tubulin, synaptophysin, or NF200.

Also within the invention is a method of promoting survival or axonal outgrowth of a retinal ganglion cell (RGC), comprising contacting the RGC with the population of persistent fetal vasculature neural progenitor cells described above. A method of conferring neuroprotection to a retinal ganglion cell in a subject is also within the invention. Neuroprotection is conferred by administering to an ocular tissue comprising a RGC a purified IGF-1 or a purified cell expressing an increased level of IGF-1. Preferably, the cell secretes IGF-1. For example, the cell comprises a vector containing a coding sequence encoding human IGF-1 or a neuroprotective fragment thereof.

The candidate subject to be treated has been diagnosed with a degenerative disease of an eye. For example, the degenerative disease comprises glaucoma, ischemic optic neuropathy, optic neuritis, or inherited mitochondrial optic neuropathies. The methods are suitable for any subject that has been characterized as suffering from or at risk of developing a neurodegenerative disease of an eye, e.g., degenerative disease or disorder of the inner retina.

A purified population of human persistent fetal vasculature neural progenitor cells comprising an heterologous or exogenous nucleic acid encoding a neuroprotective polypeptide such as an exogenous IGF-1 encoding nucleic acid or expressing/secreting a neuroprotective IGF-1 polypeptide is also within the invention. The IGF-1 comprises the full mature protein or a fragment that possesses a neuroprotective activity of the full-length protein. An exemplary human IGF-1 protein is described in GenBank: CAA01955.1, CAA01954.1 (GI:1247519), or P05019 (IGF1_HUMAN, showing molecule processing, regions/domains, and disulfide bond locations; last modified Apr. 18, 2012).

For example, IGF-1 precursor protein comprises amino acid residue 1-119, and the mature peptide comprises amino acid residues 15-84 of the sequence shown below. Fragments include any peptide that is less than the full length IGF-1, e.g., a fragment is less than 119 residues or less than the 69 amino acids, e.g., an IGF-1 fragment comprises 10, 20, 25, 30, 40, 50, 60, 65, 70, 75, 80, 90, 100, 110, 115 amino acids. IGF-1 activity of fragments is tested using known methods.

1 malclltfts satagpetlc gaelvdalqf vcgdrgfyfn kptgygsssr rapqtgivde

61 ccfrscdlrr lemycaplkp aksarsvraq rhtdmpktqk evhlknasrg sagnknyrm (SEQ ID NO: 25)

Exemplary IGF-1 encoding nucleic acid sequences include NM_{—}202494.2, NM_{—}202470.2, NM_{—}202468.2, NM_{—}005716.3, or NM_{—}202469.2. For example, such a population of cells is intravitreally transplanted into a subject to confer a clinical benefit.

Also within the invention is the use of a persistent fetal vasculature neural progenitor cell for drug discovery, wherein the cell comprises a nucleic acid encoding a reporter gene. For example, screening assays, e.g., high throughput screening assays, are carried out by contacting the cells expressing a reporter gene (e.g., encoding a detectable gene product such as a green fluorescent protein (GFP), TDtomato, or any number of other reporter gene products known in the art) with a test compound, e.g., a candidate drug, and measuring level of expression of the reporter gene (transcript or gene product) as a read-out for a specific cellular activity. Alternatively, the cells are interrogated by employing a “live-dead” reporter assay.

All polynucleotides and polypeptides of the invention are purified and/or isolated. As used herein, an “isolated” or “purified” nucleotide or polypeptide is substantially free of other nucleotides and polypeptides. Purified nucleotides and polypeptides are also free of cellular material or other chemicals when chemically synthesized. Purified compounds are at least 60% by weight (dry weight) the compound of interest. Preferably, the preparation is at least 75%, more preferably at least 90%, and most preferably at least 99%, by weight the compound of interest. For example, a purified nucleotides and polypeptides is one that is at least 90%, 91%, 92%, 93%, 94%, 95%, 98%, 99%, or 100% (w/w) of the desired nucleic acid or polypeptide by weight.

Purity is measured by any appropriate standard method, for example, by column chromatography, thin layer chromatography, or high-performance liquid chromatography (HPLC) analysis. The nucleotides and polypeptides are purified and used in a number of products for consumption by humans as well as animals, such as companion animals (dogs, cats) as well as livestock (bovine, equine, ovine, caprine, or porcine animals, as well as poultry). A purified or isolated polynucleotide (ribonucleic acid (RNA) or deoxyribonucleic acid (DNA)) is free of the genes or sequences that flank it in its naturally-occurring state. For example, the DNA is a cDNA. “Purified” also defines a degree of sterility that is safe for administration to a human subject, e.g., lacking infectious or toxic agents.

Heterologous DNA or heterologous nucleic acid refers to a DNA molecule or a nucleic acid, or a population of DNA molecules or a population of nucleic acids, that do not exist naturally within a given host cell. DNA molecules heterologous to a particular host cell may contain DNA derived from the host cell species (i.e. endogenous DNA) so long as that host DNA is combined with non-host DNA (i.e. exogenous DNA). For example, a DNA molecule containing a non-host DNA segment encoding a polypeptide operably linked to a host DNA segment comprising a promoter is considered to be a heterologous DNA molecule. Conversely, heterologous DNA can comprise an endogenous structural gene operably linked with an exogenous promoter. A heterologous protein is a protein that is expressed by the host cell and encoded by the heterologous. nucleic acid.

Publications, U.S. patents and applications, Genbank/NCBI accession numbers, and all other references cited herein, are hereby incorporated by reference.

### BRIEF DESCRIPTION OF THE DRAWINGS

FIGS. 1A-L are a series of photographs showing characterization of neural progenitor cells derived from persistent fetal vasculature (NPPFV) cells. Fundus view of a subject with persistent fetal vasculature (PFV) shows a whitish membrane in the anterior vitreous behind the lens (A, insert). Neurospheres of liberated NPPFV cells in subsequent passage (A). Neurospheres spread on fibronectin-coated plates (B). Co-expression of nestin (green fluorescence) and Pax6 (nuclear red fluorescence) in freshly dissected PFV membrane indicated the presence of neural progenitors (C). Immunostaining of typical markers expressed by undifferentiated NPPFV cells (D-H) at passage 2. Incorporation of Brdu (red nuclear fluorescence) and β-III-tubulin (green fluorescence) indicates that NPPFV cells have distinct populations. Proliferative NPPFV cells uptake Brdu have smaller morphology and fewer projections (I). Cultured NPPFV cells differentiated at passage 2 in a retinoic acid (RA) enriched conditioned-medium and developed mature neuronal morphological appearance including a round soma and extensive neurites (J). Expression of β-III-tubulin (K) and NF200 (L) in RA-differentiated NPPFV cells. Scale bar: 100 μm.

FIG. 2 is a bar graph showing gene expression profiles of undifferentiated NPPFV and human retinal progenitor cells (hRPC) by the real-time qRT-PCR at passage 5. Abbreviations: NES, nestin; RCVRN, recoverin; RHO, rhodopsin; hRPC, human retinal progenitor cell; P5, passage 5. *p<0.05 and **p<0.01.

FIGS. 3A and 3C are bar graphs and FIG. 3B is a series of photographs showing the results of an evaluation of responses of NPPFV cells to neurotransmitters by labeling the calcium indicator dye Fura-2. (A) The percentage of responding NPPFV cells after stimulation with different neurotransmitters at a concentration of 0.1 M. (B) Pseudo-color images of representative NPPFV cells before and shortly after the applications of different neurotransmitters such as Ach (acetylcholine), DOPA(dihydroxyphenylalanine), or GABA (gamma aminobutyric acid). The scale, e.g., rainbow scale from blue to violet, indicates the gradual change from low to high level of [Ca²⁺]ᵢ. (C) The peak [Ca²⁺]ᵢ of responding NPPFV cells after stimulation with different neurotransmitters at a concentration of 0.1 M. **p<0.01.

FIGS. 4A-C are photographs showing survival and migration of transplanted eGFP-expressing NPPFV cells (A-B) and hRPCs (C) to the retina of adult C57BL/6 mice. NPPFV cells survived well on day 7 post-transplantation and remained as discrete clumps in the host retina above the ganglion cell layer (GCL) and some cells migrated into the inner nuclear layer (INL) (A, indicated by white arrows). On day 21 post-transplantation, migrations of engrafted cells were observed in the INL and outer nuclear layer (ONL) (B). The engrafted hRPCs (eGFP labeled) survived well and clustered onto the GCL at week 3 post-transplantation but few cells migrated into the host retina (C). Scale bar: 20 μm.

FIGS. 5A-E are a series of photographs showing integration and differentiation of the engrafted NPPFV cells in the host retina. Immunohistochemical examination revealed that the engrafted NPPFV cells can differentiate into retinal ganglion-like neurons and form synaptic connections within the host inner retina after modulating the host retinal microenvironment by treating with RA. We observed that some engrafted cells migrated in the retina and integration into the GCL at week 3 post-transplantation (Merge, A) by immunolabelling with GFP (green) and β-III-tubulin (red). We also saw that glial reactivity significantly increased in host retina but not in the PFV cells by immunostaining with GFAP (red) after transplantation at week 3 (B). Integration of NPPFV cells into the GCL was found after 3 weeks RA treatment; the engrafted cells displayed retinal ganglion-like phenotype with β-III-tubulin expression (C, indicated by the white arrows) and a typical area covered in the white box in (C) is shown in (D) at higher magnification. Synaptophysin (red) indicated that the connection had formed between the transplanted NPPFV cells and the host inner retina (E). RGC, retinal ganglion cell; GCL, ganglion cell layer; INL, inner nuclear layer, ONL, outer nuclear layer.

FIGS. 6A-B are a photograph and a diagram, respectively. (A) Transplantation of hNPPFVs into the murine model of pigmentary glaucoma (DBA/2J mice). Representative section-RGCs (beta III-tubulin, red) that are direct contact with GFP-expressing hNPPFVs (arrow) survive the elevated pressure in the DBA/2J mouse eye (between the two *) while distant RGCs are lost (arrowheads). (B) The map of the vector expressing IGF-1 and IGFBPL1 genes for transfection in hNPPFVs.

FIGS. 7A, B, D, E, and F are photographs and FIG. 7C is a bar graph showing the results of transfection into hNPPFVs. GFP (A) and TDtomato (B) were used as controls with relatively high transfection rates (16% and 14%, respectively). The transfection rate of IGFBPL1 (D) was much lower than that of IGF-1 (E) (9% and 22%, respectively). A limited number of cells (4%) were transfected with both IGF-1 and IGFBPL1 (F). The quantified transfection rate is summarized in panel C.

FIGS. 8A-B are bar graphs and 8C-D are photographs of an electrophoretic gel showing the quantification of expression of transfected gene in hNPPFVs. Expression of IGF-1 and IGFBPL1 was quite high (Ct value>15 compared to control Tdtomato or GFP) in transfected hNPPFVs. The mRNA expression of IGF-1 (A) and IGFBPL1 (B) are shown. RT-PCR products are shown in Panel C: IGF-1 (bands 3-4); IGFBPL1 (bands 7-8). The controls without transfection (bands 1 and 5); empty vector transfection-band 2 (Tdtomato) and band 6 (GFP). (D) the internal control of GAPDH.

FIG. 9A is a photograph of an electrophoretic gel and FIGS. 9B-C are line graphs showing expression of genes in hNPPFVS after transfection. Western blot analysis indicates significant expression of IGF-1 (band 2-3 in panel A) and relatively high expression of IGFBPL1 (band 6-7 in panel A) in transfected hNPPFVs. The empty vector (bands 1 and 8) and the control without transfection (bands 4 and 8) do not significantly express these gene. ELISA analysis confirms that transfected hNPPFVs secrete IGF-1 and IGFBPL1 (B-C).

FIGS. 10A-E are photographs and FIG. 10F is a graph showing that IGF-1 promoted RGC survival and axonal outgrowth in vitro. B6 mouse RGCs were co-cultured with IGF-1 transfected hNPPFVs for three days. The LIVE/DEAD assay indicates that IGF-1 significantly promotes RGC survival (A-B and F) and axonal outgrowth (E-F) (both P<0.05). The empty vector (TDtomato) does not show the same effect (F and C).

FIGS. 11A-D are photomicrographs showing immunocytochemistry confirmation of successful transfection of tdTomato and IGF-1 into hNPPFV cells. (A) Expression of tdTomato fusion protein in hNPPFV cells after transfection. (B) Detection of tdTomato fusion protein. (C) Transfection of IGF-1-tdTomato protein in hNPPFV cells. (D) Detection the IGF-1 moiety in transfected cells. Scale bar: 50 μm.

FIGS. 12A-D are photographs, and FIG. 12E is a line graph showing IGF-1 mRNA and protein expression in hNPPFV cells, respectively. (A) qRT-PCR indicates that mRNA of mouse igf-1 is detectable only in hNPPFV^{igf-1-tdTomato} cells but undetectable in untransfected and hNPPFV^{tdTomato} cells. (B) Western blot analysis indicates expression of tdTomato fusion protein in hNPPFV^{tdTomato} and hNPPFV^{igf-1-tdTomato} cells but not in untransfected cells. (C) IGF-1-tdTomato is expressed in hNPPFV^{igf-1-tdTomato} cells but not in untransfected hNPPFV and hNPPFV^{tdTomato} cells. (D) The merge of (B) and (C) confirms the expressed band from hNPPFV^{igf-1-tdTomato} cells. (E) ELISA array detection of secreted IGF-1 in the conditioned culture medium of hNPPFV cells. The relative absorbance at 450 nm is very stable; brown curve depicts control cells and the green curve represents hNPPFV^{tdTomato} cells. The blue curve indicates the IGF-1-tdTomato concentration gradually increased from day 1 to day 3 post-transfection and reached its peak expression on day 3 (indicated by a pink arrow, top of panel). Secretion of IGF-1-tdTomato gradually fell after day 3, but remained slightly higher than 20 ng/ml at day 7 (indicated by a green arrow, right side of panel). Red curve is the standard curve showing the relative absorbance at 450 nm from serial concentrations (10-250 ng/ml) of mouse IGF-1.

FIGS. 13A-F are photographs, and Figs. G-H are bar graphs showing the survival rate and neurite outgrowth of RGC co-cultured with transfected hNPPFV cells. (A) The survival rate of RGCs co-cultured with hNPPFV^{tdTomato} cells. (B) The survival rate of RGCs co-cultured with hNPPFV^{igf-1-tdTomato} cells. β-III Tubulin stained co-cultured cells indicates that neurites were rarely observed in RGCs co-cultured with hNPPFV^{tdTomato} cells (c) but neuritis with branches were frequently observed in RGCs co-cultured with hNPPFV^{igf-1-tdDomato} cells (D-F). (G) The survival rate of RGCs co-cultured with IGF-1 (right box) was significantly higher than that of untransfected (left box) and co-cultured with hNPPFV^{tdTomato} cells (middle box); there was no difference on the survival rate of the RGCs co-cultured with untransfected and hNPPFV^{tdTomato} cells. (H) The length of neurites of RGCs co-cultured with hNPPFV^{igf-1-tdTomato} cells (right box) was significantly longer than that of untransfected (left box) and hNPPFV^{tdTomato} cells (middle box); there was no difference between untransfected and hNPPFV^{tdTomato} cells. (I) hNPPFV^{igf-1-tdTomato} cells also enhances the neurite branching (right bar), which was not observed in untransfected and hNPPFV^{tdTomato} cells (left and middle bar). (J) There was a slight negative association between neurite length and neurite number, suggesting that neurites were longer in RGCs with a single neurite but shorter in RGCs with three neurites. The boxes in (G) and (H) represent the 0.25, median and 0.75 quantiles. On either side of the box, the whiskers extend to the minimum and maximum. The detailed index of each transfection is presented in (J). The red dash line in each box is the mean value. **<0.001. Scale bar: 200 μm (A-B) and 100 μm (C-F).

FIGS. 14A-F are photographs, and FIGS. 14G-H are bar graphs showing that IGF-1 antagonists significantly blocked the function of IGF-1 on enhancing survival and neurite outgrowth of RGCs. RGCs were co-cultured with transfected hNPPFV cells. Inhibitors were added in the medium at their optimized concentrations (H-1356, 40 μg/ml; NBI-31772, 10 μM; IGF-1R antibody, 1:250). (A-F) β-III Tubulin staining indicates that the antagonists of IGF-1 significantly inhibited neurite outgrowth in mRGCs in different co-culture conditions. (G) The survival rate of RGCs was not different among the treatment groups. (H) IGF-1 antagonists inhibited neurite outgrowth of RGCs and no difference was found in the length of neurites compared to untransfected control. Boxes represent the 0.25, median and 0.75 quantiles. On either side of the box, the whiskers extend to the minimum and maximum. The dashed line in each box represents the mean value. Scale bar: 50 μm.

### DETAILED DESCRIPTION

Persistent fetal vasculature (PFV) is a potentially serious developmental anomaly in human eyes, which results from a failure of the primary vitreous and the hyaloid vascular systems to regress during development. Fibrovascular membranes harvested from subjects with PFV contain neural progenitor cells (NPPFV cells). A neural progenitor cell type was isolated from human persistent fetal vascular tissue. In this condition, the hyaloidal vascular system that nourishes the developing lens fails to regress, leaving a whitish membrane in the anterior vitreous behind the lens. This tissue contains neural progenitor cells. Studies were therefore carried out to examine whether these NPPFV cells exhibit characteristics of neuronal progenitor cells (such as antigenic and genetic profiles) and whether they were capable of differentiating into retinal neurons. To explore whether NPPFV cells have potential for cell-based inner retinal transplantation therapy, NPPFV cells were intravitreally transplanted into adult C57BL/6 mice and their integration and differentiation in the inner retina examined.

NPPFV cells highly express neuronal progenitor markers [nestin (e.g., NCBI Reference Sequence: NP_{—}006608.1; GENBANK Accession NM_{—}006617.1), Pax6 (e.g., GENBANK Accession AAX56950.1), and/or Ki67 (e.g., GENBANK CAA46520.1, NP_{—}001139438.1., NM_{—}001145966.1., NP_{—}002408.3. NM_{—}002417.4.)] as well as retinal neuronal markers [β-III-tubulin (e.g., GENBANK NP_{—}001184110.1., NM_{—}001197181.1., NP_{—}006077.2. NM_{—}006086.3.) and/or Brn3a (e.g., GENBANK NP_{—}006228.3., NM_{—}006237.3.)]. In the presence of retinoic acid and neurotrophins, these cells acquire a neural morphological appearance in vitro, including a round soma and extensive neurites, and express mature neuronal markers [β-III-tubulin and/or NF200 (e.g., NP_{—}066554.2., NM_{—}021076.3.)]. Further experiments, including real-time qRT-PCR to quantify characteristic gene expression profiles of these cells and Ca²⁺ imaging to evaluate the response to stimulation with different neurotransmitters, indicated that NPPFV cells resemble a more advanced stage of retinal development and show more differentiation toward inner retinal neurons rather than photoreceptors. To explore the potential of inner retinal transplantation, NPPFV cells were transplanted intravitreally into eyes of adult C57BL/6 mice. Engrafted NPPFV cells survived well in the intraocular environment in presence of an immunosuppressant such as rapamycin, and some cells migrated into the inner nuclear layer of the retina one week post-transplantation. Three weeks after transplantation, NPPFV cells were observed to some migrate and integrate in the inner retina. In response to daily intraperitoneal injections of retinoic acid, a portion of transplanted NPPFV cells exhibited retinal ganglion cell-like morphology and expressed mature neuronal markers [β-III-tubulin and synaptophysin (e.g., GENBANK NP_{—}003170.1., NM_{—}003179.2.)]. These findings indicate that fibrovascular membranes from human PFV harbor a population of neuronal progenitors are useful for cell-based therapy for degenerative diseases of the inner retina.

The following materials and methods were used to carry out the data described herein.

Isolation of Primary Cells and PFV Cell Culture.

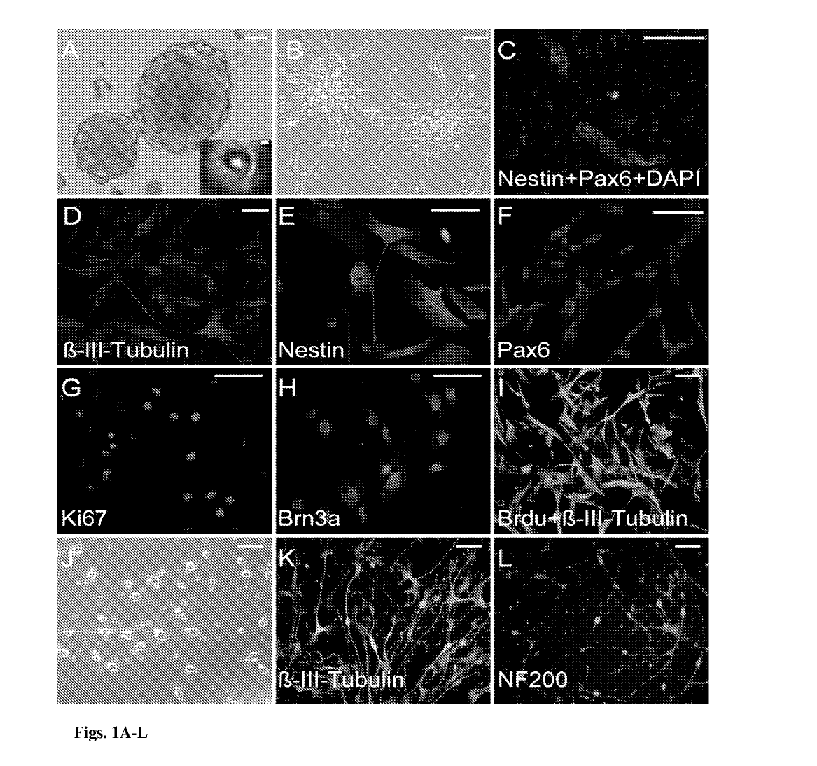

PFV membranes (FIG. 1A) were obtained from young donors during corrective surgery. Upon receipt of the PFV membrane, the tissue was placed in 1× phosphate-buffered saline (PBS) containing 3× concentration of penicillin-streptomycin, finely minced, placed in 0.1% type 1 collagenase (Invitrogen, Carlsbad, Calif., USA) and agitated for 20-30 min at room temperature. Liberated cells in the collagenase solution were collected, forced through a 70 μm sieve, centrifuged and plated in X-vivo medium (Lonza, Watersville, Md., USA) supplemented with 10% fetal bovine serum (FBS, Invitrogen), 1:50 B27 (Invitrogen), 1:100 N2 (Invitrogen), 10 ng/ml bFGF (Invitrogen), 20 ng/ml EGF (Invitrogen) and 50 μg/ml nystatin (Sigma, St. Louis, Mo. USA). Additional collagenase was added to the remaining tissue until all tissue was dissociated. Cells were seeded on 24 well-tissue culture plates following each cycle and incubated at 37° C. in a humidified 95% air and 5% CO₂-environment. After 24-48 hr, neurospheres were observed in the culture medium and transferred to fibronectin coated tissue culture flasks in X-vivo medium with supplements described above. Cells were fed every 2 days and passaged or frozen down 75-80% confluence. At passage 5, a subset of cells was also cultured in differentiation medium consisting of all-trans-retinoic acid (100 nM, Sigma), brain-derived neurotrophic factor (50 ng/ml, Invitrogen), ciliary neurotrophic factor (20 ng/ml, Invitrogen), nerve growth factor (20 ng/ml, Invitrogen) and 10% FBS. NGF (e.g., GENBANK NP_{—}002497.2., NM_{—}002506.2). BDNF (e.g., NP_{—}001137277.1., NM_{—}001143805.1., NP_{—}001137278.1., NM_{—}001143806.1., NP_{—}001137279.1., NM_{—}001143807.1., NP_{—}001137280.1., NM_{—}001143808.1., NP_{—}001137281.1., NM_{—}001143809.1., NP_{—}001137282.1., NM_{—}001143810.1. NP_{—}001137283.1., NM_{—}001143811.1., NP_{—}001137284.1., NM_{—}001143812.1., NP_{—}001137285.1., NM_{—}001143813.1., NP_{—}001137286.1., NM_{—}001143814.1., NP_{—}001137288.1., NM_{—}001143816.1., NP_{—}001700.2, NM_{—}001709.4., NP_{—}733927.1., NM_{—}170731.4., NP_{—}733928.1., NM_{—}170732.4., NP_{—}733929.1., NM_{—}170733.3., NP_{—}733930.1., NM_{—}170734.3., NP_{—}733931.1., NM_{—}170735.5.). NT-3 (e.g., GENBANK NP_{—}001096124.1., NM_{—}001102654.1., NP_{—}002518.1., NM_{—}002527.4.). NT-4 (e.g., GENBANK NP_{—}006170.1., NM_{—}006179.4.). CNTF (e.g., GENBANK NP_{—}000605.1., NM_{—}000614.3.). IGFBP-1 (e.g., GENBANK NP_{—}000587.1., NM_{—}000596.2.).

Cells then were examined for morphological features, antigenic profiles, gene expression and neurotransmitter receptors.

Immunocytochemical Examination.

NPPFV cells were seeded on slide-chambers (VWR, Batavia, Ill., USA) and cultured in a differentiation-conditioned medium for at least 7 days or in normal growth medium for examining their antigenic profiles. Cells were fixed for 10 min in 4% buffered paraformaldehyde, washed in PBS and blocked with 10% goat serum (Vector, Burlingame, Calif., USA)/PBS solution for 30 min before immunocytochemical staining. Primary antibodies [including β-III-tubulin (Sigma), nestin (Sigma), Pax6 (Chemicon-Millipore, Billerica, Mass., USA), Ki67 (Vector), Brn3a (Chemicon), Brdu (ABCAM, Cambridge, Mass., USA), NF200 (neurofilament-200, Sigma), recoverin (Chemicon), RPE-65 (Chemicon), CRALBP (ABCAM), GFAP (Sigma), α-SMA (ABCAM), FSP1 (Sigma), CD31 (ABCAM) were diluted in 2% goat serum/PBS solution to appropriate concentrations for incubating cells at 4° C. overnight. Cells were rinsed with PBS and incubated with a secondary antibody consisting of either Cy3 (Chemicon 1:500) or FITC (Chemicon 1:300) at room temperature for 30 min on the following day. After rinsing in PBS, slides were mounted with Vectashield mounting medium containing DAPI (Vector) and visualized under an inverted fluorescence microscope (Olympus 1X51). All immunocytochemical analyses were repeated 3 or more times from the cells at passage 2 to 5. Related isotype immunoglobulins were used as negative controls (Chemicon).

Evaluation of Gene Expression by Real-Time aRT-PCR.

To determine the gene profiles of NPPFV cells, real-time qRT-PCR was performed to compare designated mRNA expression of NPPFV cells with those of human retinal progenitor cells (hRPCs). hRPCs have been isolated from human fetal tissue and well characterized in vitro. The isolation procedures for those cells are known (Klassen, H. J.; Ziaeian, B.; Kirov, I. I.; Young, M. J.; Schwartz, P. H. Isolation of retinal progenitor cells from post-mortem human tissue and comparison with autologous brain progenitors. J. Neurosci. Res. 77:334-343; 2004). Retinas of donors with an estimated age of 18 weeks of gestation were cut into small pieces on a dry petri dish under a tissue culture hood and enzymatically digested in a sterile container at 37° C. with periodic removal of supernatants and refilling with fresh digestion solution. Harvested cells from the supernatants after centrifugation were resuspended in cell-free retinal progenitor-conditioned medium, and then cells were transferred to fibronectin-coated tissue culture flasks containing fresh media. In the present study, both cell types (NPPFV and RPC) from passage 0 were cultured in the same medium after the original isolation from tissue samples to eliminate any possible influence from different culture conditions on their respective gene expressions. The mRNA expression of various transcription factors and retinal specific proteins has been detected by PCR in early passages of cultured hRPCs. Therefore, hRPCs can serve as a well-established control for NPPFV cells in real-time-qPCR experiments. Total RNA was extracted from NPPFV cells and hRPCs (both at passage 5) using an RNA isolation kit (Qiagen, RNeasy Mini Kit). To ensure samples without genomic DNA contamination, total RNA was treated with DNase (Qiagen, RNase-Free DNase Set) and cDNA was synthesized using a Synthesis Kit (Bio-rad). Total cDNA (1 μl) was loaded in each well, mixed with PCR master mix (TaqMan Universal, Applied Biosystems, Foster City, Calif.) and pre-designed primers (IDT, San Diego, Calif.) for Pax6, nestin, ATOH7, recoverin, rhodopsin, SNAP25, STXBP1, RAPSN and THY1, respectively (Listed in Table 1). The procedure for real-time qRT-PCR included 2 min at 50° C., 15 min at 95° C., followed by 40 cycles of 15 s at 95° C., 30 s at 55° C., and 30 s at 72° C. (ABI PRISM 7900 HT; Applied Biosystems). Expression (evaluated as fold change for each target gene) was normalized to glyceraldehyde-3-phosphate dehydrogenase (a housekeeping gene) in hRPCs following the well-established delta-delta method (Schmittgen, T. D.; Livak, K. J. Analyzing real-time PCR data by the comparative C(T) method. Nat. Protoc. 3(6):1101-1108; 2008.). All assays were performed in triplicate. In addition, a non-template control was included in the experiment to estimate DNA contamination of isolated RNA and reagents.

TABLE 1
Applied Primers for Real-time qRT-PCR

| | | | SEQ | | SEQ |
|---|---|---|---|---|---|
| | GenBank | Sense Primers | ID | Anti-sense Primers | ID |
| Genes | Accession | (5′ to 3′) | NO: | (5′ to 3′) | NO: |
| PAX6 | NM_000280 | AGGTATTACGAGACTGGCTCC | 1 | TCCCGCTTATACTGGGCTATTT | 11 |
| NES | X65964 | GAAACAGCCATAGAGGGCAA | 2 | TGGTTTTCCAGAGTCTTCAGTG | 12 |
| | | A | | A | |
| ATOH7 | NM_145178 | ACTGCCTTCGACCGCTTAC | 3 | CAGAGCCATGATGTAGCTCAG | 13 |
| RCVRN | NM_002903 | CTCCTTCCAGACGATGAAAAC | 4 | GCCAGTGTCCCCTCAATGAA | 14 |
| | | A | | | |
| RHO | NM_000539 | GCTTCCCCATCAACTTCCTCA | 5 | AGTATCCATGCAGAGAGGTGTA | 15 |
| | | | | G | |
| SNAP2 | NM_003081 | ATGCCCGAGAAAATGAAATG | 6 | AGCATCTTTGTTGCACGTTG | 16 |
| 5 | | | | | |
| STXBP | D63851 | CGCCTCATCATTTTCATCCT | 7 | CATTGTTGGAGCCTGATCCT | 17 |
| 1 | | | | | |
| RAPSN | NM_005055 | CTGCACTGTCTGAGCGAGAG | 8 | ACCTGAGGTGGAAGATGTGG | 18 |
| THY1 | NM_006288 | TGAAGGTCCTCTACTTATCCG | 9 | GCACTGTGACGTTCTGGGA | 19 |
| | | C | | | |
| GAPDH | NM_002046 | ATGGGGAAGGTGAAGGTCG | 10 | GGGGTCATTGATGGCAACAATA | 20 |

Abbreviations: PAX6-paired box 6, NES-nestin, ATOH7-atonal homolog 7, RCVRN-recoverin, RHO-rhodopsin, SNAP25-synaptosomal-associated protein (25 kDa), STXBP1-syntaxin binding protein 1, RAPSN-receptor-associated protein of the synapse, THY1-Thy-1 cell surface antigen, GAPDH-glyceraldehyde-3-phosphate dehydrogenase.

TABLE 2
Antigenic Profiles of Cultured NPPFV Cells

| | Specification | Undifferentiated | Differentiated |
|---|---|---|---|
| Antigens | of Cell Types | Cells | Cells |
| β-III-tubulin | Neuron | + | + |
| nestin | Neuronal progenitor | + | − |
| Pax6 | Neuronal progenitor | + | − |
| Ki67 | Proliferative cell | + | − |
| Brn3a | Neuron | + | Equivocal |
| Brdu | Proliferative cell | + | − |
| | (S phase) | | |
| NF200 | Neuron | Equivocal | + |
| recoverin | Photoreceptor | − | − |
| RFE-65 | Retinal pigment | − | − |
| | epithelium | | |
| CRALBP | Müller glial cell | − | − |
| GFAP | Glial/astrocytic cell | − | − |
| α-SMA | Myofibroblast | − | − |
| FSP1 | Fibrocyte/fibroblast | − | − |
| CD31 | Endothelial cells | − | − |

Estimation of Intracellular Ca²⁺ in NPPFV Cells.

To investigate the neurotransmitter receptors expression on NPPFV cells, Ca²⁺ imaging was performed by loading cells with the ratiometric Ca²⁺ sensitive Fura-2 dye. Cells were incubated at 37° C. for 30 min in X-vivo medium (containing 3% FBS, 5 μM Fura-2 tetra-acetoxymethyl ester, 8 μM pluronic acid F127 and 250 μM sulfinpyrazone). Cells were washed in modified Mg²⁺-free Hank's balanced salt solution (HBSS, 2.6 mM CaCl₂, 15 mM HEPES [pH 7.4] and 250 μM sulfinpyrazone). Five types of neurotransmitters, γ-aminobutyric acid (GABA), glutamate (Glu), glycine (Gly), dopamine (Dopa) and acetylcholine (Ach) were dissolved in modified HBSS at a concentration of 0.1 mM. The largest dynamic range for Ca²⁺-dependent fluorescence signals is obtained by excitation at 340 nm and 380 nm, and detecting their ratio of emission fluorescence intensities at around 510 nm. From this ratio, the concentration of intracellular Ca²⁺ ([Ca²⁺]ᵢ) can be estimated, using dissociation constants that are derived from calibration curves. By using the ratio of fluorescence intensities produced by excitation at two wave lengths, factors such as uneven dye distribution and photo bleaching are minimized (InCyt Im2TM Ratio Imaging System, Cincinnati, Ohio). The change of [Ca²⁺]ᵢ was calculated from the difference between the peak Ca²⁺ concentration evoked by each agonist and the control value of HBSS (before the addition of agonist). At least 80 cells were selected for analyzing the change of [Ca²⁺]ᵢ evoked by each agonist, averaged over three repetitions.

Inner Retinal Transplantation.

For studying their potential for inner retinal transplantation, NPPFV cells or hRPCs were intravitreally injected to 20 and 10 C57BL/6 mice, respectively. Animals were maintained in standard animal facility. To trace the transplanted cells, NPPFV cells and hRPCs were infected with an AAV2 or retrovirus vector harboring EGFP (HGTI, Boston, Mass.) following the instructions of each transfection kit, respectively. Mice (4-6 weeks of age) were deeply anesthetized with an intraperitoneal injection of ketamine (120 mg/kg) and xylazine (20 mg/kg) and pupils were dilated with 0.5% topical tropicamide. Dissociated GFP-positive cells (1×10⁵ cells/2 μl) were suspended in HBSS were vitreous cavity of the eye through a glass micropipette connected to a 10 μl Hamilton syringe via polyethylene tubing. Sham-injected mice received HBSS without cells. All experimental animals received injection in one eye and the other eye was used as an untreated control. A small puncture in the cornea (paracenthesis) was used to reduce the intraocular pressure during the transplantation surgery. Rapamycin (2 mg/kg·day) (LC laboratories, Woburn, USA) was administered to all surgical animals to ensure the survival of the xenograft. To facilitate the differentiation of engrafted NPPFV cells, 10 mice were given intraperitoneal injections of retinoic acid (RA, 2 mg/kg·day, Sigma) starting one day prior to transplantation and continuing until termination of the experiment. Of the 10 mice transplanted with hRPCs, 5 received Rapamycin, another 5 received Rapamycin and RA. Animals received terminal anesthesia on week 1 or week 3 after transplantation and the eyes were harvested after intracardial perfusion with 4% paraformaldehyde in PBS. Eyes were cryosectioned at 10 μm and examined under a Lecis TSC SP5 confocal microscope to evaluate the expression and location of different markers. Anti-GFP antibody (1:100, ABCAM, USA) was used to enhance the fluorescence of prelabled-GFP, while anti-β-III-tubulin (1:500, Sigma, USA), anti-GFAP (1:300, Sigma, USA) and anti-synaptophysin (1:100, DAKO, USA) antibodies helped to assess the differentiation of the transplanted cells. To estimate the survival and migration of grafted cells, serial sections on 4 randomly selected eyes that had undergone NPPFV transplantation were performed. Every 10^{th} serial section was counted, measuring 10 μM in thickness as to avoid redundancy of cell counts.

Statistical Analysis.

Statistical analysis was performed using the SPSS 12.0 software package. Results are expressed as mean±SEM (standard error of mean).

Differences between groups were compared by using One-Way ANOVA followed up with Tukey's test or t test, as appropriate, and two-tailed p values are reported.

hNPPFV Progenitors for Retinal Transplantation

Immunocytochemical characterization of NPPFV cells was carried out as described above. Following surgical dissection of clinical PFV membranes (funduscope of PFV subject, FIG. 1A insert) and subsequent isolation of cell contents, cells were collected and seeded in neural-supporting medium. As the cells were passaged, neurospheres began to emerge from the cultured NPPFV cells (FIG. 1A). Neurospheres were spread onto fibronectin-coated plates from which some cells grew long and slender projections shortly after the transfer (FIG. 1B). Immunofluorescent imaging of PFV tissue confirmed that some cellular elements in PFV co-expressed Pax6 (red nuclei) and nestin (green filaments) (FIG. 1C), indicating that PFV tissue contains some neural progenitors. Using immunochemical staining for nestin and/or Pax6, about 0.2% (±0.16%) of resident cells in PFV membrane were neuronal progenitors (FIG. 1C). Although the population of those cells is relatively small, the cells are able to stably maintain their progenitor phenotype and normally proliferate in cultural conditions. After being expanded up to 20 passages, characteristic markers of neuronal progenitors and retinal neurons were confirmed in the cultured NPPFV cells, including β-III-tubulin, nestin, Pax6, ki67 and Brn3a (FIG. 1D-H).

Further incorporation of Brdu and β-III-tubulin staining revealed a subpopulation of proliferating NPPFV cells that were smaller in size with fewer projections than their non-dividing counterparts in the same batch (FIG. 1I). After treatment with RA-enriched medium, NPPFV cells exhibited a typical neuronal morphological appearance, including a round soma and extensive neurites (FIG. 1J). Immunolabelling confirmed that differentiated NPPFV cells only expressed β-III-tubulin (FIG. 1K) and NF200 (FIG. 1L) and not the other aforementioned markers. NPPFV cells were also investigated to exclude any possible contamination with other cell types by screening for the following markers: photoreceptors (recoverin), retinal pigment epithelia (RPE65), astroglia (CRALBP, GFAP), myofibroblasts (α-SMA), fibroblasts (FSP1) and endothelia (CD31). But none of these markers were detected in NPPFV cells (Summarized in Table 2).

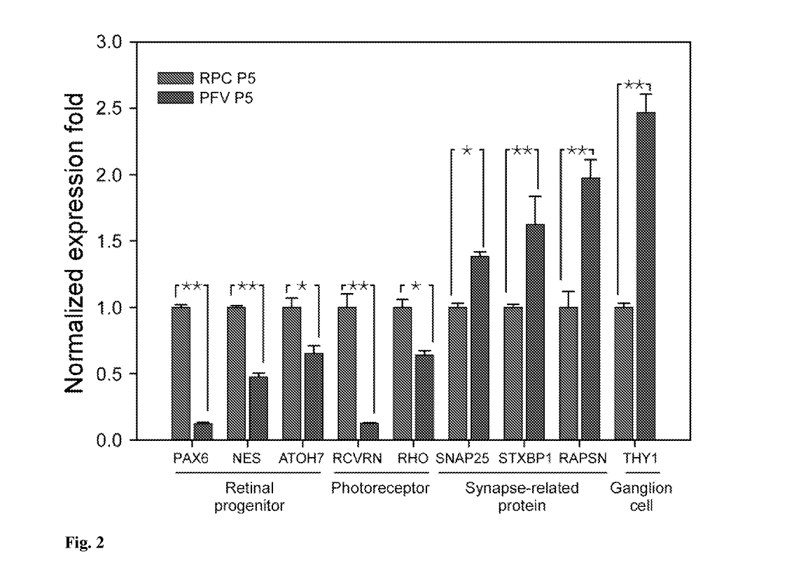

Gene expression profiles of NPPFV cells were evaluated. Characteristic markers of retinal progenitors (Pax6, nestin and ATOH7), photoreceptors (recoverin and rhodopsin), synapse-related proteins (SNAP25, STXBP1 ad RAPSN) and retinal ganglion cell (THY1) were selected to establish the gene expression profile of NPPFV cells. Real-time qRT-PCR revealed lower expression of retinal progenitor and photoreceptor markers in NPPFV cells than in hRPCs (t test, all p<0.05) (FIG. 2). However, mRNA levels of the synapse-related proteins and of THY1 (a mature retinal ganglion cell marker) were significantly higher in NPPFV cells than in hRPCs (t test, all p<0.05) (FIG. 2). These observations indicated that NPPFV cells are a type of tissue-specific progenitor in the retina with higher potential for differentiating toward inner retinal neurons (such as retinal ganglion cells) rather than photoreceptors.

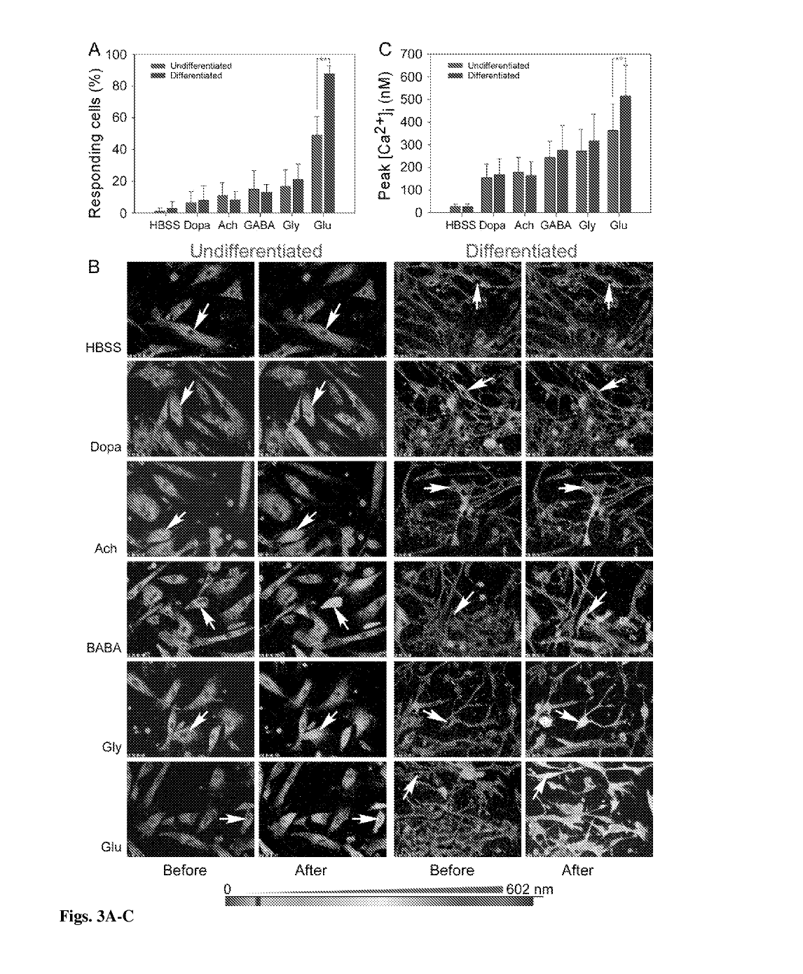

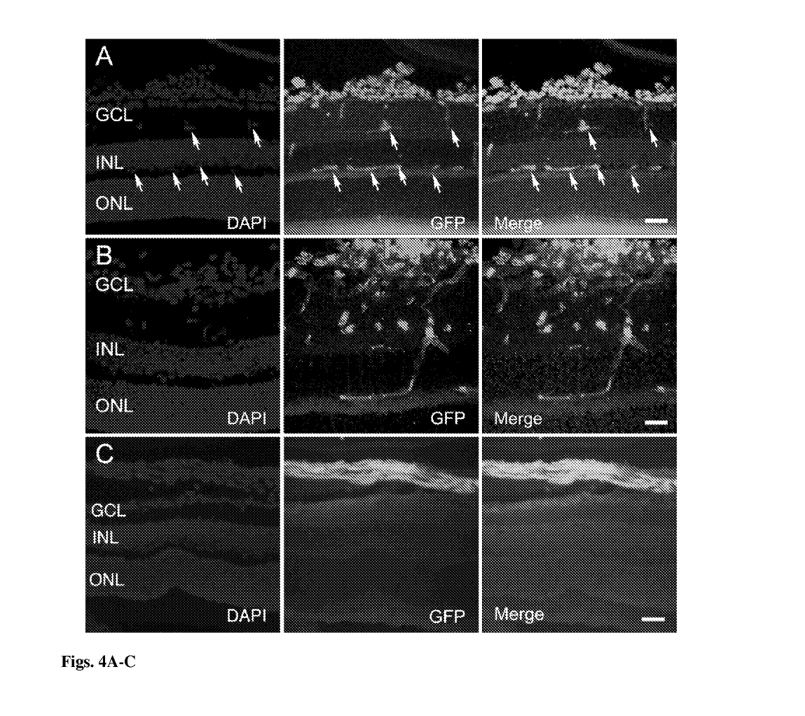

Relative levels of intracellular calcium were determined. The relative number of undifferentiated and differentiated NPPFV cells was determined, and their Ca²⁺ concentration was tested using Fura-2 dye after stimulation with different neurotransmitters. A very small portion of undifferentiated and differentiated NPPFV cells responded to Dopa (6.41±7.14% vs. 7.89±9.18%), Ach (10.96±8.12% vs. 8.23±5.41%), GABA (14.94±11.73% vs. 12.99±4.86%) and Gly (16.67±10.62% vs. 21.05±9.72%) and no significant difference was found in each group (t test, all p>0.05). However, Glu stimulation elicited a large response in undifferentiated and differentiated NPPFV cells (48.98±11.67% vs. 87.88±4.97%) compared to other transmitters (One-Way ANOVA, p<0.01). In addition, Glu evoked a response in a higher proportion of differentiated NPPFV cells than undifferentiated cells (t test, p<0.01) (FIG. 3A).

Some cells that responded to neurotransmitter stimulation were selected for measurements of relative [Ca²⁺+]ᵢ (indicated by white arrows in FIG. 3B). A limited number of NPPFV cells responded to stimulation with Dopa, Ach, GABA and Gly. Interestingly, the elicited [Ca²⁺]ᵢ of these cells was also quite low, with little or no difference between differentiated and undifferentiated NPPFV cells. Glu elicited a significant increase in the concentration of Ca²⁺ in undifferentiated and differentiated NPPFV cells compared to the rest of the neurotransmitters examined (One-Way ANOVA, all p<0.01) (FIG. 3C). Glu stimulation elicited a higher [Ca²⁺]ᵢ in differentiated than in undifferentiated cells (t test, p<0.01, FIG. 3C).

Survival and migration of transplanted NPPFV cells and hRPCs was studied.

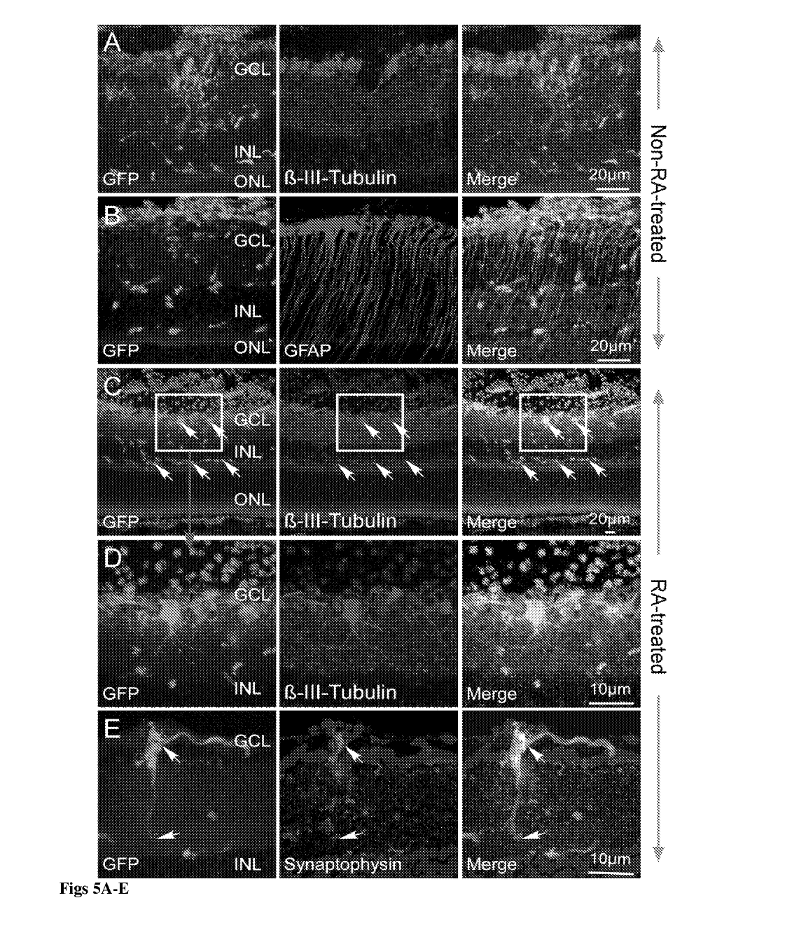

Transplanted NPPFV cells were tracked in vivo by their expression of EGFP, which was enhanced by anti-GFP antibody staining. DAPI was used to identify the nuclei of live cells. Generally, engrafted cells pooled together in the posterior vitreous and remained as discrete clumps adjacent to the ganglion cell layer (GCL) at week 1 post-transplantation (FIG. 4A). Some transplanted cells were observed in the inner retina and among these cells, some exhibited migratory-like morphological features in the outer or inner plexiform layers (indicated by white arrows, FIG. 4A). A number of engrafted cells were observed at 3 weeks post-transplantation in the inner and outer retina (FIG. 4B). EGFP-labeled NPPFV cells could be observed in retinal tissues extending up to the outer nuclear layer (ONL) (FIG. 4B). Approximately 3.51+2.60% (range, 0.83%-6.22%) grafted cells appeared to have migrated in the retina, but many surviving NPPFV cells still adhered to the ganglion cell layer (FIG. 4-5). As some of the cellular segments may not have been clearly stained by immunohistochemistory, the percentage cited above may be an underestimate.

Some migratory NPPFV cells exhibited characteristically neural cell morphology, including long and slender projections. Although some migration of engrafted NPPFV cells was found in host retina, the anatomic structure of the retina appeared morphologically normal with distinct structural layers as revealed by DAPI labeling (FIG. 4B). In addition, EGFP labeled hRPCs were also injected into the posterior vitreous of adult C57BL/6 using the same procedures as NPPFV cells. Engrafted hRPCs survived well and clustered above the GCL at 3 weeks post-transplantation; however, few engrafted hRPCs could be found in the inner retina (FIG. 4C). To our knowledge, this is the first observation that engrafted human progenitor cells can survive and migrate into the inner retina of adult animals through intravitreal transplantation.

Integration and differentiation of transplanted NPPFV cells was evaluated. Typical markers of glial cells and mature neurons were applied for checking the neuronal behaviors of the NPPFV cells in the host retina. Immunolocalization of β-III-tubulin (red fluorescence) expression of engrafted cells indicated that a small number of the engrafted cells had already merged in the GCL at 3 week post-transplantation (FIG. 5A). Similarly, glial reactivity, indicated by GFAP (red fluorescence), was significantly increased in the host retina, but few NPPFV cells were GFAP positive (FIG. 5B). Glial activation seems to be a significant obstacle for migration of engrafts, as some NPPFV cells were observed in a gliosis.

Post-transplanted differentiation into mature retinal neurons or glial cells was not clearly observed in the engrafted cells. Induction of the differentiation of the transplanted cells was attempted by modulating the retinal microenvironment with intraperitoneal treatment of RA. After 3 weeks of treatment, β-III-tubulin was detected in these GFP-positive engrafts that naturally integrated into the GCL and inner nuclear layer of the host retina (white arrows, FIG. 5C). Some integrated NPPFV cells in the GCL presented as mature retinal ganglion-like cells, such as a round soma and extensive neurites (FIG. 5D). Furthermore, immunostaining for synaptophysin (a functional marker of mature neurons) revealed that synaptic connections had formed between the NPPFVs and the inner retinal cells (indicated by white arrows, FIG. 5E). The percentage of differentiated NPPFVs may be relatively low, but they were not rare. These findings indicated that the NPPFV cells could differentiate into retinal ganglion-like neurons in the host retina in response to environmental neural differentiation cues such as RA.

### Neural Stem Cell Therapy in the Eye

If any possible therapeutic effects of replacing lost RGCs with transplanted stem cells are to be achieved for degenerative inner retinal conditions, it is essential that a suitable cell type be chosen for transplantation and a more bio-compatible method be used to facilitate the integration and differentiation of engrafted cells in the host tissue. Glaucoma is not be the only inner retinal condition benefits from restoration of RGCs. RGC protection and/or replacement is applicable to other diseases that result in the death or dysfunction of RGCs, such as ischemic optic neuropathy, optic neuritis, and inherited mitochondrial optic neuropathies.

Patient-derived or donor neural progenitor cell type isolated from human persistent fetal vascular tissue provide a solution to cell therapy problems of previous approaches. In PFV, the hyaloidal vascular system that nourishes the developing lens fails to regress, leaving a whitish membrane in the anterior vitreous behind the lens. As described herein, this tissue contains neural progenitor cells. NPPFV cells exhibited characteristics of neuronal progenitor cells (such as antigenic and genetic profiles) and were found to be capable of differentiating into retinal neurons.

The findings from immunohistochemical examination, real-time qRT-PCR and Ca²⁺ imaging confirm that NPPFV cells exhibit characteristics of neuronal progenitor cells and were induced to differentiate into mature neurons in vitro. As an appropriate source of stem cells is fundamental to transplantation therapy, NPPFV cells can be easily grown from the surgically dissected tissue from human subjects with PFV, cryopreserved, and passaged for relatively long periods of time, e.g., 10, 20, 25, 50, 100 or more passages. This suggests that these cells are useful for clinical application. Intravitreal transplantation of NPPFV cells demonstrated that they survive well and migrate into the inner retina. Furthermore, modulation of the retinal microenvironment by intraperitoneal injection of RA significantly increases the differentiation of engrafts into retinal ganglion-like mature neurons, which highlights the utility of facilitating engraft integration by modulating the in vivo microenvironment. These results indicate that neural progenitor cells derived from the PFV membrane are well suited for cell-based therapy for inner retinal neurodegenerative diseases, such as glaucoma and that the therapeutic effects of these cells in animal models of glaucoma and other retinal injury models demonstrate their ability to provide neuroprotection for RGCs.

Although numerous recent studies have highlighted the possibility of applying stem cell-based therapy for retinal degenerative diseases, multiple fundamental problems must be resolved before it can be used clinically Few of these methods can be directly transferred to clinical application, considering the obvious side-effects on other retinal resident cells. Therefore, there has been a pressing need to find a suitable cell type and establish some clinically feasible methods if any cell-based therapy is to be considered for inner retinal diseases in the future. The compositions and methods described herein provide a solution to the drawbacks and inadequacies associated with prior stem cell-based approaches for treatment of retinal disease.

As described herein, neural progenitor cells derived from human PFV membranes were successfully cultured and characterized. Although the population of those cells in PFV membrane was relatively small, they stably maintain the same characteristics as progenitor cells and normally proliferate in cultural conditions (FIG. 1A-B). Therefore, it was not difficult to harvest enough cells for therapeutic use.

Considering the derivation and location of PFV membranes, studies were carried out to determine whether these membranes contained retinal progenitor cells. Repeated screening for different antigenic markers indicated that undifferentiated NPPFV cells exhibit characteristics of neural progenitors instead of other retinal cell types (Table 2). However, these NPPFV cells could be differentiated in vivo into a retinal ganglion cell-like morphology and express neuronal markers. High expression of β-III-tubulin was observed in undifferentiated NPPFV cells as well. β-III-tubulin is usually considered to be one of the earliest neuron-associated cytoskeletal markers, and plays a significant role in neuritogenesis and cell motility during retinal development. Some studies have revealed that expression of β-III-tubulin could be found in immature neurons of the fetal retina and different neuronal progenitor cells. Some pre-migratory neuroblasts in the postnatal human brain also express β-III-tubulin. Therefore, high expression of β-III-tubulin in undifferentiated NPPFV cells indicates that these progenitors exhibit a migratory phenotype, which facilitates their migration and integration after transplantation.

hRPCs have been isolated from human fetal tissue and well characterized in vitro. The mRNA expression of various transcriptional factors and retinal specific proteins has been detected by PCR in early passages of cultured hRPCs (1). Real-time qRT-PCR results revealed that, compared to hRPCs, NPPFV cells expressed lower mRNA levels of retinal progenitor cell markers and photoreceptor cell markers, but higher mRNA levels of synapse-related protein and THY1 (a mature RGCs marker) (FIG. 2). hRPCs only migrated and integrated in ONL of the host retina and expressed the mature rod marker rhodopsin after subretinal space injection in mice suggesting that hRPCs could be differentiated along the photoreceptor lineage. Therefore, given the different derivations of these two cell types, NPPFV cells may reside in a later developmental stage than hRPCs, and NPPFV cells have a greater potential predisposition to differentiate into inner retinal neurons compared to photoreceptors.

The neurotransmitter profile of NPPFV cells was examined using Ca²⁺ imaging analysis, which is widely used to evaluate neural precursors. Unlike neurons from the central nervous system, differentiated NPPFV cells exhibited limited responses to Dopa, Ach, GABA and Gly, but robust responses to Glu (FIG. 3). Glu, GABA and Gly are major neurotransmitters for conducting visual signals in the vertebrate retina. Although neurotransmitter content of several lateral synapses are yet to be determined, the vast majority of synapses mediating center inputs to bipolar cells and ganglion cells are glutamatergic, and those mediating lateral synapses are GABAergic (horizontal cells and amacrine cells) and glycinergic (amacrine cells). Ca²⁺ imaging examinations indicated that the majority of differentiated NPPFV cells exhibited a receptor profile similar to glutamatergic neurons. The data indicate that NPPFV cells exhibit a neuronal progenitor phenotype and have the potential to differentiate along the ganglion cell lineage.

Cell migration into the uninjured inner retina was observed as early as 1-week and became more significant at 3 weeks post transplantation. This observation indicated that these cells adopted an energetic migratory phenotype after transplantation. As supported by the literature, the number of cells that generally penetrate the retina is usually modest. Millions of cells may be transplanted. However in the examples described here, transplants have been limited to 50,000 cells, which is relatively a small number. Yet the yield of cellular penetration is similar to those studies using many more cells. Some engrafted cells exhibited long and slender projections across the inner plexiform layer. This is the first observation of migration and integration of human cells into uninjured inner retina of adult mice. Of interest, transplanted NPPFV cells seemed to be restricted from migrating into the ONL. Few engrafted cells were found in the outer retina, although sufficient migration was observed in the inner retina. Experiments on subretinal transplantation of NPPFV cells revealed that transplanted cells pooled around the injection site and were restricted from migrating through the ONL. Given that no physical barrier has been reported on the inner side of ONL, it may be possible that the microenvironment in the ONL is inhibitory for NPPFV cell migration. Studies have shown that when progenitor cells are transplanted intravitreally into the adult rodent eye, they do not generally penetrate the retinal barriers and reside on the retinal surface. In order for these cells to penetrate the retina, the retinal barriers need to be broken down. This is achieved by either creating a break in the internal limiting membrane or disturbing Müller/astroglia with glutamate agonists such as α-aminoadipate. The present methods have a distinct advantage over other approached, because NPPFV cells can penetrate the intact inner retina.

Although a plenty of engrafted NPPFV cells were seen in the host retina, the recipient retinal tissue appears morphologically unremarkable with clear structural layers. It is possible that the immunosuppressant effect of rapamycin may have protected the retina, at least partly, from serious xenograft rejection, as severe inflammation usually leads to damage of host tissue after transplantation. The inhibitory effect of rapamycin on mammalian targets of rapamycin pathway may play a role in protecting retina from injuries.

The host retinal environment plays a vital role in neural differentiation of transplanted cells, and additional modulation of retinal environment has been proven to improve migration, integration and differentiation of engrafted cells. Retinoic acid (RA) is an established signaling molecule that is involved in neuronal patterning, neuronal differentiation and the maintenance of the differentiated state of adult neurons and neural stem cells. Embryonic stem cells, hematopoietic stem cells and neural stem cells can be diverted down the neural differentiation pathway using combinations of RA and growth factors, or neurotrophins, which have also been implicated in vivo for their ability to enhance survival and replace lost neurons in the adult brain.

Studies were carried out to test the possibility of inducing differentiation of transplanted NPPFV cells using by RA in vivo. Transplanted NPPFV cells were found to be integrated into the host GCL and exhibited a retinal ganglion cell-like morphology after RA treatment. Expression of synaptophysin was observed, albeit in low frequency, between the connections of differentiated NPPFV cells and host retinal neurons. Similar connections were observed between the engrafted cells and host cells. Although the estimation of the number of differentiated NPPFV cells was very low by visualization of EGFP expression, it is possible that they were underestimated due to the variation of EGFP expression.

However, successful application of RA has proven that it is possible to modify the in vivo environment to enhance the integration and differentiation of transplanted cells in the inner retina. Although NPPFV cells have the potential to differentiate into RGC-like cells, this does not mean that native (in situ) NPPFV cells nested within the retrolental membrane are able to directly migrate from the retrolental membrane into the INL toward areas of retinal pathology without therapeutic intervention. There are many organic and microenvironmental factors that can affect the fate shift and migration of stem cells. Regardless, RA regulates NPPFV cell differentiation in vivo increase the yields and efficiency of inner retina migration and differentiation of NPPFV cells.

Transfection of IGF-1 and IGFBPL1 in Neuronal Progenitor Cells from Human Persistent Fetal Vascular for Neuroprotection

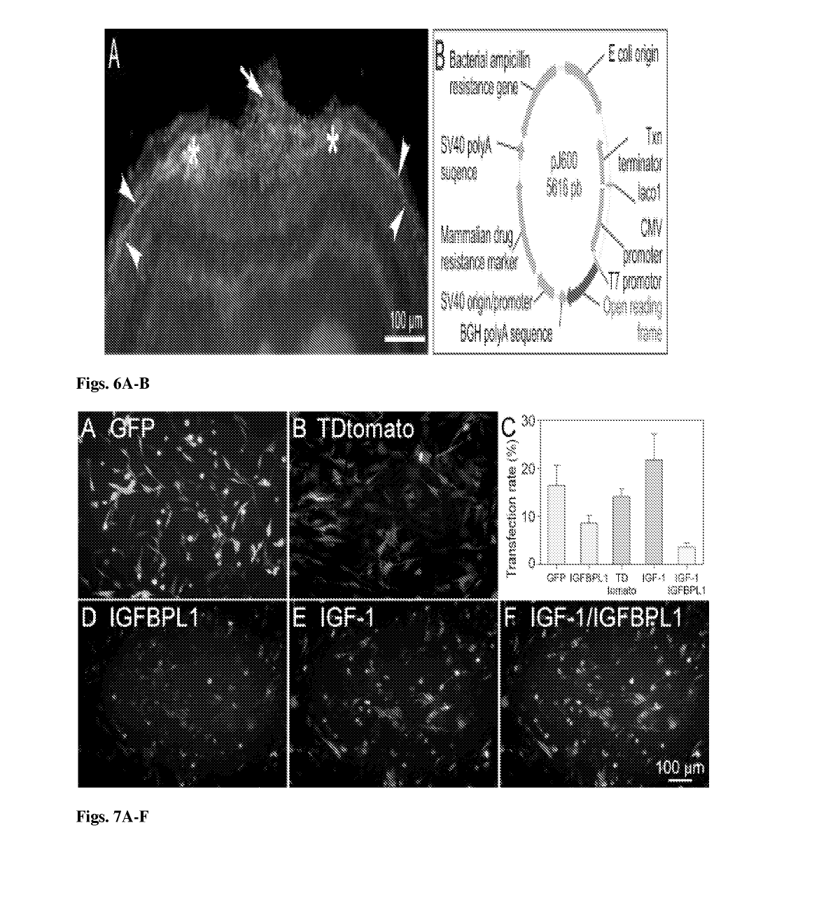

As described above, cells isolated from human persistent fetal vasculature membranes are replete with retinal progenitor cells (hNPPFVs), which differentiate into retinal neurons. In DBA/2J pigmentary glaucoma mice, transplanted hNPPFVs attach and integrated into the inner retinal layer and the optic nerve head (FIG. 6A). Moreover, resident RGCs located in proximity of hNPPFVs survive, while distant RGCs perish. Based on these observations, studies were undertaken to determine whether hNPPFVs produce neuroprotective factors that allow survival of some RGCs in the ocular hypertensive environment of the DBA/2J mouse. Experiments were carried out to determine whether transfection of a known neuroprotective factor, insulin-like growth factor-1 (IGF-1) and insulin-like growth factor binding protein-like 1 (IGFBPL1) could confer more robust and global neuroprotection to host RGCs

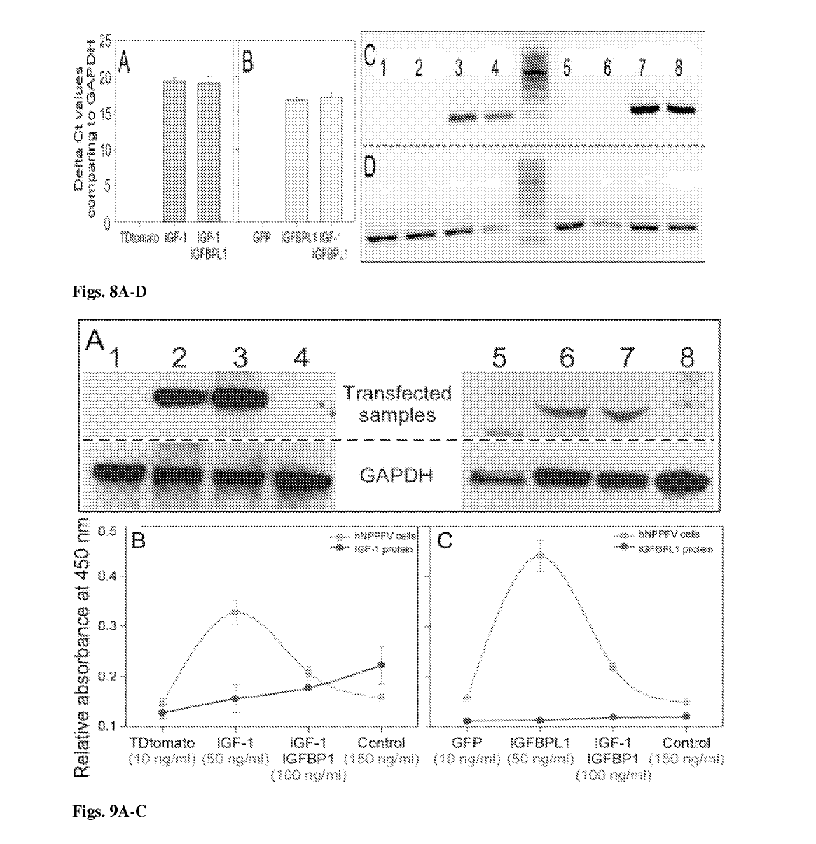

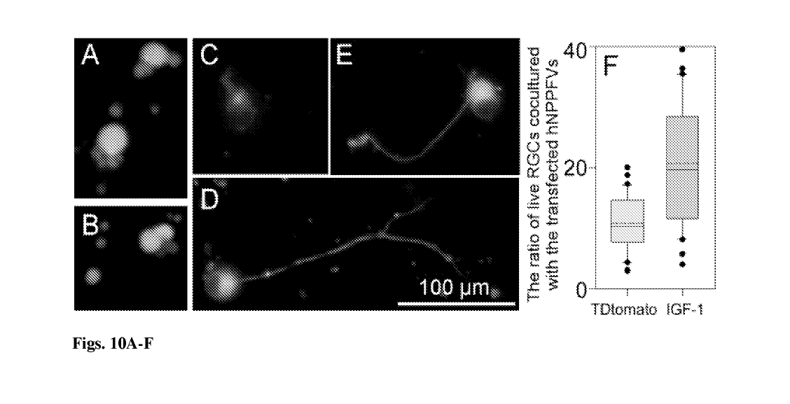

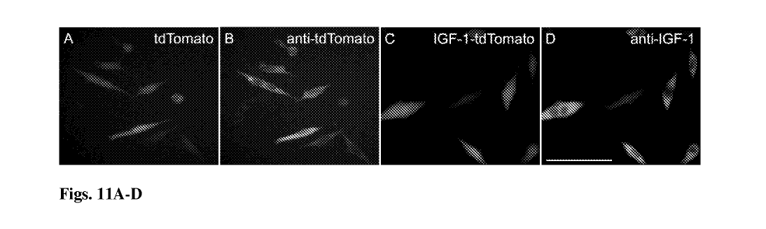

hNPPFVs were transfected with different plasmids at the concentration of 20 ng/ml. Expression of IGF-1-Tdtomato (FIG. 6B) and IGFBPL1-GFP (FIGS. 7A-F) was examined three days after transfection. These data indicate successful transfection of IGF-1 and IGFBPL1 to hNPPFVs. FIGS. 8A-D show the quantification of the transfected IGF-1 and IGFBPL1 in hNPPFVs by measuring mRNA levels, and FIGS. 9A-C show expression of IGF-1 and IGFBPL1 in hNPPFVs after the transfection at the protein level. ELISA analysis confirms that the expressed recombinant IGF-1 and IGFBPL1 are secreted by the cells. As shown in FIGS. 10A-E, IGF-1 was found to promote RGC survival and axonal outgrowth.

The data from these studies indicated that 1) IGF-1 and IGFBPL1 can be successfully transfected into hNPPFVs; 2) transfected hNPPFVs significantly express and secrete both IGF-1 and IGFBPL; and 3) IGF-1 significantly promotes RGC survival and axonal outgrowth. Thus, increasing IGF-1 and/or IGFBPL1 levels is useful for neuroprotection and promotion of survival and growth of retinal neurons.

### Example 1

### Neuroprotection of IGF-1 on Retinal Ganglion Cells

As was described above, neuronal progenitor cells from human persistent fetal vasculature incorporate into the retinal ganglion cell (RGC) layer after transplantation. To study whether hNPPFV cells could function as serogate vehicles for local delivery of neuroprotective factors, the effects of IGF-1 were evaluated. RGCs co-cutured with IGF-1-transfected hNPPFV cells displayed significantly enhanced survival, neurite extension and branching, while selective inhibitors of IGF-1 signaling blocked these responses. The findings indicate that transfected hNPPFV cells abundantly deliver IGF-1 and significantly invigorate neuronal survival. The results also indicate that local cell-based delivery of selected neurotrophic factors to protect and rehabilitate host RGCs under disease conditions.

Cells have generally been used as a carrier to load a specific gene to scale up or scale down the expression in a signaling pathway or gene therapy. However, cells used as vehicles in previous studies, unlike hNPPFV cells, have not demonstrated integration and differentiation in host retina after intravitreal transplantation. Based on the advantages of the differentiative potential of hNPPFV cells in the retina and the neurosupportive properties of IGF-1, studies were carried out to test whether hNPPFV cells could be used as candidate vehicles loading igf-1 for continued local delivery of igf-1 in the host retina, whether hNPPFV cells could be stably transfected to express sustained levels of biologically active IGF-1, and whether increase production and secretion of IGF-1 could confer global neuroprotection on RGCs. In this example, igf-1 was cloned into a plasmid carrying a fluorescence reporter gene (tdTomato) to generate fluorescent fusion proteins. The coding sequences of igf-1-tdTomato or tdTomato alone were inserted into a pJ603-neo plasmid backbone. pJ603-neo^{igf-1-tdTomato} generates a fusion protein with tdTomato tagged to the C-terminus of IGF-1. A pJ603-neo^{tdTomato} vector generating tdTomato protein was used as a control vector. Transfected cells were studied under co-culture conditions with B6 mouse RGCs and evaluated for their effects on neuronal morphology, apoptosis and neurite growth of RGCs. In addition, we also utilized two IGF-1 antagonists (H-1356 and NBI-31772) with altered affinities for IGF-1 and IGF-1 binding protein, and an antibody to IGF-1R in order to address the neuroprotective mechanisms of IGF-1 signaling pathway. Fragments of IGF-1 that are neuroprotective are identified using the assays described below.

The following materials and methods were used to generate the data described in this example.

Transfection of hNPPFV Cells

hNPPFV cells were thawed from a cell bank. They were previously isolated from human persistent fetal vasculature and were cultured according to established protocols. The coding sequences of igf-1-tdTomato or tdTomato were inserted into a pJ603-neo plasmid backbone (DNA2.0, Menlo Park, Calif.). pJ603-neo^{igf-1-tdTomato} generates a fusion protein with tdTomato tagged to the C-terminus of IGF-1, and pJ603-neo^{tdTomato} generates a tdTomato protein alone (used as control vector). Optionally, Gaussia luciferase signal peptide connected at the N-terminus was used to improve IGF-1 expression and secretion. The plasmids were transfected into DH5α Competent E. Coli cells, expanded, and purified using the EndoFree Plasmid Maxi Kit (Qiagen, USA). hNPPFV cells were seeded onto 6-well plates at 1×10⁵ cells/well. The next day, the culture medium in each well was replaced with 1 ml the transfection complex (60 μl Lipofectamine 2000 (Invitrogen); 240 μl plasmid (about 650 ng/ml); and serum-free X-vivo medium). The transfection medium was replaced with regular growth medium after a 5 hr incubation at 37° C. (95% O₂, 5% CO₂).

### Immunocytochemical Analysis

hNPPFV cells were transfected with pJ603-neog^{igf-1-tdTomato} or pJ603-neo^{tdTomato} in 96-well plates using the same procedure as described above. Two days post-transfection, cells were fixed in 4% paraformaldehyde (20 min), washed in 1×PBS and blocked with the blocking buffer (Li-Cor, Odyssey, Lincoln, Nebr.) at room temperature (30 min). Cells were incubated in primary antibody solution at 4° C. overnight and then rinsed in PBST three times (10 min of each) before being incubated with a secondary antibody solution at room temperature (30 min). After rinsing in PBST twice (10 min in total), cells were visualized under an inverted fluorescence microscope (Olympus 1X51). Primary antibodies used in this study were goat anti-mouse IGF-1 (1:400, B&D systems, MN) and rabbit anti-RFP (1:300, Rockland, Pa.). Secondary antibodies were goat anti-rabbit Cy3 (Chemicon, 1:500) and donkey anti-goat FITC (Chemicon, 1:500).

### Quantitative RT-PCR

Transfected hNPPFV cells previously seeded onto 6-well plates were lysed at 48 hr after the transfection and total RNA was extracted using RNeasy Plus Mini Kit (Qiagen, Valencia, Calif.). cDNA was synthesized using the SuperScript® III First-Strand Synthesis System (Life Technologies, USA). 0.5 μl cDNA, 1 μl pre-designed primers of IGF-1 (forward 5′-agatgcactgcagtttgtgtgtgg-3′ SEQ ID NO: 21, reverse 5′-tctacaattccagtctgtggcgct-3′ SEQ ID NO: 22) or GAPDH (forward 5′-ggcctccaaggagtaagacc-3′SEQ ID NO: 23, reverse 5′-aggggtctacatggcaactg-3′ SEQ ID NO: 24), 8.5 μl RNase/DNase-free water and 10 μl KAPA SYBR® FAST reaction buffer (Kapa Biosystems, USA) were loaded into 96-well white PCR plates. qRT-PCR was performed using the Roche LightCycler 480 (Roche LC480, Roche Applied Science) using the following program: 5 min at 99° C., followed by 40 cycles of 15 s at 94° C., 15 s at 59° C., and 15 s at 72° C. Relative amount of igf-1 mRNA expression was normalized to gapdh mRNA (evaluated as ct values) using the well-established delta-delta method. All assays were performed in triplicate. A non-template control was included in the experiment to estimate DNA contamination of isolated RNA and reagents.

### Western Blot

Two days post-transfection, hNPPFV cells were lysed to extract total protein using 1×RIPA buffer (Cell Signaling) containing 1 mM phenylmethylsulfonyl fluoride, 1× Protease inhibitor cocktail and 1×EDTA (Thermo Scientific, Rockkford, Ill.). Protein lysates were loaded on 4-20% precise pre-casted PAGE gels (Thermo Scientific, Rockkford, Ill.) and subjected to electrophoresis. Gels were semidry transferred to nitrocellulose membranes (Bio-Rad, Hercules, Calif.) for immuno-blot analysis. Membranes were blocked with blocking buffer for 1 hr at room temperature, incubated with primary antibodies including goat anti-mouse IGF-1 (B&D systems, MN, 1:400) and rabbit anti-GAPDH (1:400, Rochland, Pa.) diluted with the blocking buffer and PBST (volume rate 1:1) overnight (4° C.). Membranes were washed twice in PBST (10 min of each), incubated with secondary antibodies (1:3,000, anti-goat IRDye 800CW and anti-rabbit IRDye 680LT, Li-Cor, Odyssey, Lincoln, Nebr.) for 1 hr, and washed twice in PBST (10 min of each). Fluorescent protein bands were visualized on the Odyssey Infrared Imaging System (Odyssey, Lincoln, Nebr.). After imaging, membranes were briefly striped in stripping buffer (Thermo Scientific), and rinsed in PBST before incubating with rabbit anti-RFP (1:300, Rockland, Pa.) and rabbit anti-GAPDH (1:400) diluted with the blocking buffer and PBST (volume rate 1:1) at 4° C. overnight, followed by secondary antibody incubation. Protein bands were imaged the next day as described above.

Detection of IGF-1 Secretion from Transfected hNPPFV Cells

Secretion of IGF-1 (as component of the IGF-1-tdTomato fusion protein) from hNPPFV^{igf-1-tdTomato} or hNPPFV^{tdTomato} cells was detected using ELISA assay. A 96-well Elispot plate was coated by sodium carbonate buffer (50 μl/well) overnight at 4° C. Conditioned culture medium from hNPPFV^{igf-1-tdTomato} or hNPPFV^{tdTomato} cells (collected on day 1, 3, 5 and 7) and recombinant mouse IGF-1 protein (10-250 ng/ml) were used to coat the Elispot plate overnight (4° C.). After being briefly rinsed, the wells were filled with the blocking buffer (200 μl/well, 10% FBS in 1×PBS) at room temperature for 2 hr. The wells were washed twice with 1×PBS and incubated with goat anti-mouse IGF-1 (1:400) at 4° C. overnight. Wells were washed with 1×PBST three times (5 min each time) and then incubated with chick anti-goat HRP-conjugated secondary antibody (Sigma, 1:5000). Wells were washed twice with 1×PBST and once with 1×PBS. TMB (3,3′,5′5-tetramethylbenzidine) was added to the wells and incubated in dark area for 15-20 min. The reaction was stopped with H₂SO₄ (2 M) and the plate was quickly read with a Flox4 microarray reader system at OD 450 nm.

### RGC Dissociation and Purification

P0 B6 mice were euthanized with CO₂ and the retinas were dissected out from the eyecups in cold Hank's buffer (Life Technologies) containing 1× Penicillin-Streptomycin-Glutamine (Life Technologies). Retinas were digested in 20 U/ml papain solution containing 100 U/ml DNaseI at 37° C. for 5-15 min; the reaction was stopped with 5 mg/ml ovomucoid protease inhibitor containing 5 mg/ml albumin. Retinas were gently triturated to obtain a single cell suspension, and cells were washed once and re-suspended in 800 μl washing buffer (0.5% BSA, 2 mM EDTA in 1×PBS). RGCs were isolated using Thy1.2 (CD90.2) microbeads and MACS® magnetic separation system (Miltenyi Biotech) following the manufacturer's instructions. RGCs were centrifuged and re-suspended in RGC culture medium (Neurobasal-A medium supplemented with 25 μM L-glutamic acid, 1 mM L-glutamine, 100 U/ml penicillin, 100 μg/ml streptomycin, 1×B-27, 5 μg/ml insulin, 50 ng/ml BDNF, 50 ng/ml CNTF and 1 μM forskolin) (Life Technologies).

Co-Culture of RGCs with Transfected hNPPFV Cells

hNPPFV cells transfected with pJ603-neo^{igf-1-tdTomato} or pJ603-neo^{tdTomato} plasmids were seeded onto cell culture inserts (0.4 μm pore size, BD Falcon) and incubated. On day 3, RGCs were seeded onto 12-well plates pre-coated with Poly-D-Lysine (Millipore, 0.1 mg/ml) and merosin (Millipore, 5 μg/ml), and 200 μl of RGC culture medium was added into each well; culture medium in the inserts containing transfected hNPPFV cells was replaced with 200 μl RGC culture medium before being transferred to the wells. The plates were maintained in an incubator (37° C., 95% O₂ and 5% CO₂) for three days before being subjected to survival and neurite outgrowth assays. In some experiments, the following reagents were added into the culture medium immediately after RGCs were seeded: IGF-1 receptor antagonist (H-1356, Bachem, 40 μg/ml) which acts as a competitive inhibitor of the IGF-1 receptor, IGFBP (IGF-binding protein) inhibitor NBI-31772 (Millipore, 10 μM), which disrupts the binding of IGF-1 with all six IGFBPs, and a blocking antibody to IGF-1 receptor (IGF-1R, 1:250, R&D Systems).

### RGC Survival Assay and Neurite Outgrowth Assay

Co-culture inserts and culture media were removed after 3 days; RGCs were washed with 1×PBS and stained with CalceinAM and EthD-1 (LIVE/DEAD® Viability/Cytotoxicity kit, Life Technologies) for 40 min at room temperature. Images of 4-6 view fields were randomly selected throughout each well under an Olympus inverted fluorescence microscope. Live and dead cells were counted using ImageJ 1.46 (National Institutes of Health, Bethesda, Md.) and survival rates were calculated as live cells/live+dead cells×100%. RGCs in some other wells were fixed with 4% paraformaldehyde for 15 min and then incubated with rabbit anti-mouse β-III Tubulin (Millipore, 1:800) overnight (4° C.) and incubated with goat anti-rabbit Cy3 (1:800) for 1 hr. Images were acquired with an Olympus inverted fluorescence microscope. Neurite lengths were measured using ImageJ.

### Statistical Analysis

Statistical analysis was performed using the Sigma Plot. Results are expressed as mean±SD (standard deviation). Differences between groups were compared using Independent Sample t-test. Two-tailed P values <0.05 were defined as significant.

hNPPFV^{igf-1-tdTomato} cells secrete high levels of IGF-1

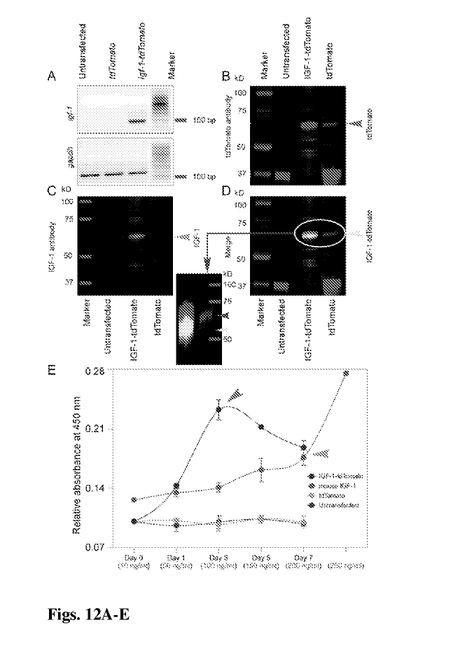

To investigate the neuroprotective effects of IGF-1 on RGCs and to evaluate whether hNPPFV cells can be used as effective vehicles for IGF-1 delivery, igf-1-tdTomato was introduced into hNPPFV cells by transfecting the cells with plasmid carrying the igf-1-tdTomato fusion gene. In the control group, cells were transfected with a plasmid containing tdTomato. Two days after transfection, over 80% of the cells expressed red fluorescence consistent with tdTomato expression. Immunostaining of the transfected cells with antibodies against tdTomato or IGF-1 confirmed the expression of tdTomato and IGF-1-tdTomato proteins (FIGS. 11A-D).

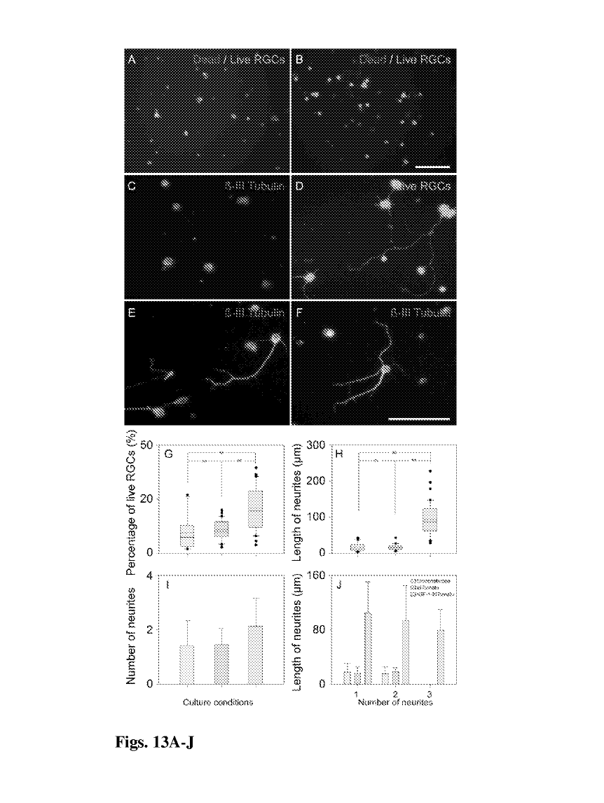

To quantify the mRNA levels of igf-1, total RNA was extracted two days post-transfection from hNPPFV^{igf-1-tdTomato} and hNPPFV^{tdTomato} cells and used for quantitative RT-PCR (qRT-PCR). High level of mouse igf-1 mRNA was detected in hNPPFV^{igf-1-tdTomato} cells. In contrast, no mouse igf-1 mRNA was detected in untransfected or hNPPFVTff^{tdTomato} cells (FIG. 12A).

To identify the expression of IGF-1-tdTomato protein in hNPPFV^{igf-1-tdTomato} cells, whole cell lysates were prepared two days after transfection and used for Western blot analysis. High levels of IGF-1-tdTomato were detected in hNPPFV^{igf-1-tdTomato} cells using antibody against IGF-1, at a molecular weight of 60 kD, agreeing with its predicted molecular weight (about 52 kD for tdTomato portion, and 7.6 kD for IGF-1 portion). TdTomato was detected using antibody against tdTomato in both hNPPFV^{igf-1-tdTomato} and hNPPFV^{tdTomato} cells (FIG. 12B), but no IGF-1 or IGF-1-tdTomato was detected in hNPPFV^{tdTomato} cells (FIG. 12C). The locations of IGF-1 and tdTomato are presented in FIG. 12D.

The levels of IGF-1-tdTomato secreted from the hNPPFV^{igf-1-tdTomato} cells were assessed by ELISA using the conditioned culture media collected from the hNPPFV^{igf-1-tdTomato} cells at 1, 3, 5 and 7 days post-transfection. Mouse recombinant IGF-1 protein with concentrations ranging between 10 ng/ml to 250 ng/ml was used to generate the standard curve (as shown in FIG. 12E; red line). The level of IGF-1-tdTomato gradually increased in the first three days and peaked on day 3 post transfection, with the concentration reaching about 225 ng/ml (blue curve in FIG. 2E) in the hNPPFV^{igf-1-tdTomato} cells group. Thereafter, the IGF-1-tdTomato levels dropped slowly, likely due to apoptotic cell death of hNPPFV^{igf-1-tdTomato} cells over time, but still remained higher than 200 ng/ml on day 7. In contrast, there was no detectable level of IGF-1 in hNPPFV^{tdTomato} or in untransfected cells (green and brown lines in FIG. 12E), which was consistent with the qRT-PCR results that no igf-1 mRNA was detected in hNPPFV^{tdTomato} or in untransfected cells. Additionally, the levels of IGF-1 naturally released by untransfected hNPPFV cells were measured in conditioned media using ELISA and found to be negligible and below the detection curve of the ELISA.

### IGF-1 Enhanced the Survival and Neurite Outgrowth of RGCs

To evaluate the effects of IGF-1 on RGC survival and neurite outgrowth, primary RGCs were co-cultured from postnatal day 0 (P0) mouse retina with hNPPFV^{igf-1-tdTomato} cells in vitro. We compared survival rates and neurite extension of RGCs co-cultured with hNPPFV^{igf-1-tdTomato} cells with RGCs co-cultured with hNPPFV^{tdTomato} or untransfected cells. The survival rate of RGCs co-cultured with hNPPFV^{igf-1-tdTomato} cells (22±13%) was significantly higher than those co-cultured with hNPPFV^{tdTomato} or untransfected cells (P<0.05; FIGS. 13A-B and FIG. 13G); the latter two had similar survival rates of 11±5% and 10±8%, respectively, with no significant statistical difference (FIG. 13G).

The average neurite length of RGCs co-cultured with hNPPFV^{igf-1-tdTomato} cells was also significantly longer than those co-cultured with hNPPFV^{tdTomato} or untransfected cells. RGCs co-cultured with hNPPFV^{igf-1-tdTomato} cells produced dramatically long neurites with average lengths of 93±45 μm (P<0.05, FIG. 13H). While RGCs co-cultured with untransfected or hNPPFV^{tdTomato} cells displayed average neurite lengths of 17±12 μm and 17±9 μm, respectively, with no significant difference (P>0.05, FIG. 13H). RGCs co-cultured with hNPPFV^{igf-1-tdTomato} cells produced more neurites on average (2±1 neurites/cell; range, 1 to 5) compared with RGCs co-cultured with untransfected or hNPPFV^{tdTomato} cells (both averaged around 1 neurite/cell, ranging from 1 to 5) (both P<0.05, FIGS. 13C-F, and 13I). RGCs co-cultured with hNPPFV^{igf-1-tdTomato} cells exhibited different axonal morphologies—some RGCs produced single, longer neurites others produced short, branching neurites. These observations indicated that is more than one population of cultured RGCs and that there may be a negative association between axonal length and branching (FIG. 13J).

Effects of Inhibitors of IGF-1 Signaling Pathway on IGF-1-tdTomato-Mediated RGC Survival and Neurite Outgrowth

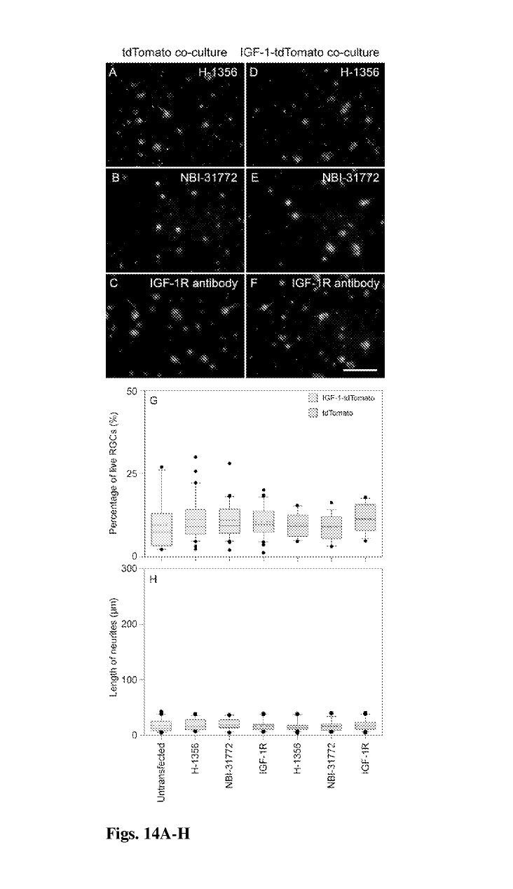

To confirm that the enhancing effects on RGC survival and neurite outgrowth in the hNPPFV^{igf-1-tdTomato} co-cultured group was attributed to activation of the IGF-1 signaling pathway mediated through the IGF-1R, we applied antagonists of the IGF-1 signaling pathway to the culture medium in the co-culture system. H-1356 is an IGF-1 analog, which competitively binds with IGF-1R and blocks IGF-1 signaling. NBI-31772 disrupts the binding of IGF-1 with all six IGF-1 binding proteins. Applying both of these inhibitors completely eliminated the effects of IGF-1-tdTomato on RGC survival and neurite outgrowth (FIGS. 14A-B, D-E). Similarly, application of the neutralizing antibody to IGF-1R also completely blocked the effects on RGC survival and neurite outgrowth mediated by IGF-1-tdTomato (FIGS. 14C and F). The survival rates and average neurite lengths of the RGCs co-cultured with hNPPFV^{igf-1-tdTomato} cells in presence of IGF-1 inhibitors or IGF-IR neutralizing antibody were no longer significantly different from those co-cultured with hNPPFV^{tdTomato} or untransfected cells (detailed comparison showing in Table 3) (FIGS. 14G-H).

TABLE 3
Repeated comparison matrix (P values) of live cell survival rate and neurite
length of RGCs co-culturing with hNPPFV^{tdTomato} or hHNPPFV^{igf-1-tdTomato} cells

| | | | tdTomato co-culture | | | IGF-1-tdTomato co-culture | | |
|---|---|---|---|---|---|---|---|---|
| | | | | NBI- | IGF-1R | | | IGF-1R |
| | Treatments | Untransfected | H-1356 | 31772 | antibody | H-1356 | NBI-31772 | Antibody |
| Survival rate | Untransfected | | 0.99 | 0.84 | 0.50 | 0.56 | 0.62 | 0.70 |
| tdTomato co- | H-1356 | 0.99 | | 0.72 | 0.22 | 0.29 | 0.32 | 0.42 |
| culture | NBI-31772 | 0.84 | 0.72 | | 0.12 | 0.17 | 0.17 | 0.23 |
| | IGF-1R | 0.50 | 0.22 | 0.12 | | 0.88 | 0.74 | 0.57 |
| | antibody | | | | | | | |
| IGF-1-tdTomato | H-1356 | 0.56 | 0.29 | 0.17 | 0.88 | | 0.86 | 0.69 |
| co-culture | NBI-31772 | 0.62 | 0.32 | 0.17 | 0.74 | 0.86 | | 0.80 |
| | IGF-1R | 0.70 | 0.42 | 0.23 | 0.57 | 0.69 | 0.80 | |
| | antibody | | | | | | | |
| Neurite length | Untransfected | | 0.86 | 0.72 | 0.79 | 0.99 | 0.82 | 0.91 |
| tdTomato co- | H-1356 | 0.86 | | 0.86 | 0.64 | 0.84 | 0.68 | 0.93 |
| culture | NBI-31772 | 0.72 | 0.86 | | 0.50 | 0.68 | 0.55 | 0.77 |
| | IGF-1R | 0.79 | 0.64 | 0.50 | | 0.77 | 0.99 | 0.66 |
| | antibody | | | | | | | |
| IGF-1-tdTomato | H-1356 | 0.99 | 0.84 | 0.68 | 0.77 | | 0.81 | 0.89 |
| co-culture | NBI-31772 | 0.82 | 0.68 | 0.55 | 0.99 | 0.81 | | 0.71 |
| | IGF-1R | 0.91 | 0.93 | 0.77 | 0.66 | 0.89 | 0.71 | |
| | antibody | | | | | | | |

### Neuronal Progenitor Cells Overexpressing IGF-1 Enhance Retinal Ganglion Cell Survival and Neurite Outgrowth

Previous studies have confirmed the essential roles of IGF-1 on neuron survival and development in the CNS. In the retina, the extended neural structure of the CNS, IGF-1 is a normal constituent playing its role in development. Invtravitreal injections of IGF-1 inhibited secondary cell death of axotomized RGCs in rats. Some in vitro and in vivo studies have showed that IGF-1 is developmentally-regulated and contributes to the visual cortex development. However, these full biological spectrums of IGF1-induced effects on RGCs have remained largely unknown. Moreover, a single intravitreal injection of IGF-1 is short-lasting, and repetitive injections of IGF-1 are needed a considered to be poorly acceptable to patients in clinic. In order to further elucidate the effects of IGF-1 on RGCs as well as to develop a less-invasive, long-lasting and more effective delivery approach, cellular system for delivering biologically active IGF-1 was developed using hNPPFV retinal progenitor cells. The biological effects of IGF-1 on mouse primary RGCs were evaluated in co-culture system, which allowed investigation of the function of IGF-1 on RGC survival and neurite outgrowth, as well as evaluation of the efficacy of using the hNPPFV cells as delivery vehicles for production of IGF-1 to RGCs in vitro. These results indicated that cell-based strategies using hNPPFVs (or NPPFVs from other species) are useful for local delivery of neurotrophic factors into the retina to prevent RGC death and promote optic nerve survival after injury or in disease conditions.

In this example, the igf-1-tdTomato plasmid was successfully transfected into the hNPPFV cells at over 80% transfection rate. qRT-PCR and Western blots confirmed the expression of the transgenes in the hNPPFV cells. ELISA experiments indicated that IGF-1-tdTomato was continuously secreted into the culture medium at high efficiency. In vivo studies of retina have shown that igf-1 mRNA localizes to the ganglion cell layer but no specific localization has been seen in eye sections. In the retina of mammals, IGF-1 plays an essential role during prematurity. Hypodevelopment of vascular growth in retina after premature birth, which is partially due to the insufficiency of IGF-1 expression, causes serve retinopathy leading to visual disorder. A recent study of teleost retina indicates that IGF-1 showed its significant function on regulating rod progenitor proliferation. The data described herein demonstrated that IGF-1 dramatically increased the survival rates and neurite outgrowth of RGCs, and that hNPPFV cells can be used as an efficient delivery vehicle to continuously provide IGF-1.

IGF-1 is primarily synthesized in the liver and plays an essential role in growth and development, and continues to have anabolic effects through adulthood. IGF-1-mediated neuroprotection may involve multiple pathways, including PI-3 kinase and MAPK pathways. The specific receptor of IGF-1 mediated its primary action, which is composed of an extracellular ligand-binding domain that controls the activity of its intracellular tyrosine kinase domain. Mature mouse retinas exhibit decreased the expression levels of p85α regulatory subunit, which was in concurrence with diminished Akt phosphorylation of PI-3 kinase pathway. Furthermore, they provided evidence of retinal and RGC basal lipid kinase activity related with upstream signaling components, such as p85 regulatory and p110 catalytic pathways. The data described herein showed that the survival rate of cultured RGC was remarkably increased in the IGF-1 enriched microenvironment But the survival rate significantly decreased when inhibitors of IGF-1R signaling pathways were applied, which was coincident with previous studies that blocking of IGF-1R signaling pathways induced interruption of survival and proliferation in mitosis-competent cells and growth of tissues. Therefore, binding of newly-secreted IGF-1 to its cognate receptor leads to the cascades of cell signaling resulting to increased survival of RGCs. As described in Example 1, the secreted IGF-1 also affected the morphology of RGCs; specifically, RGCs grew more and longer neurites than those in controls. IGF-1 was also observed to enhance arborization of the neurites in RGCs.

Visual field changes in glaucoma are believed to be caused by the loss of RGCs, although the exact cause of RGC degeneration is still unknown. The aim of neuroprotection for glaucoma therapy is to use agents that prevent or delay retinal ganglion cell death, as well as rescue and enhance regeneration of already compromised RGCs. hNPPFV cells serve as local synthesis pods for local delivery of IGF-1 as they spontaneously integrate into the host RGC and nerve fiber layers after intravitreal injection.

The neuronal progenitor cells and cell lines described herein spontaneously hone in on the RGC and nerve fiber layer and are therefore useful as vehicles for local delivery of a desired neurotrophic factor. The findings indicate that the hNPPFV^{igf-1-tdTomato} cells continuously secret IGF-1-tdTomato, and that the hNPPFV cells efficiently incorporated into the RGC layer after intravitreal transplantation and differentiate into RGC-like cells and survived in the retina over very long period. hNPPFV cells are therefore useful for efficient delivery and supply of neuroprotective factors to prevent RGC death and promote regeneration in vivo in the future.

### Example 2

### High Throughput Assay for Modulators of Neural Pathways

hNPPFV cells expressing various reporter sequences such as that described above are useful in high-throughput screening for drug discovery and related applications. hPPFVs are neurons and are thus used as substitutes for other neuronal cells in high-throughput assays. Since hNPPFV cells have RGC-like characteristics, they can be utilized in both undifferentiated and differentiated cells.

hNPPFV cells have many advantages compared to other cells used for such assays. For example, primary RGCs are difficult to culture and poorly survive culture conditions necessary for high-throughput screening. Unlike primary RGC cultures, hNPPFV cells have a prolonged survival in tissue culture and can easily accept ectopic reporter gene sequences, which function as reporters for specific cellular activity. hPPFVs may be used as substitute cells for primary RGCs. Therefore, large numbers of molecules may be assayed for neurotrophic activity in hPPFV cells using “live-dead” cells reporter assay. The “hits” can then be further tested on primary RGCs.

Another advantage is the ability to test medium spiny neurons (MSN) pathway function and screen for modulators thereof. An example is determining the intracellular signaling processing of brain neurons such as MSN, which are very difficult to culture and study in vitro. Compounds that influence certain cellular and signaling pathways in MSNs may be subjected to high-throughput study using reporter genes or fluorescent fusion constructs (similar to td-Tomato/IGF-1 described above) that are transfected into hPPFVs. These constructs encoding a detectable marker are used as reporters of cellular pathways of interest. Using the same paradigm, “hits” are then further confirmed on MSN cells.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. Genbank and NCBI submissions indicated by accession number cited herein are hereby incorporated by reference. All other published references, documents, manuscripts and scientific literature cited herein are hereby incorporated by reference.

While this invention has been particularly shown and described with references to preferred embodiments thereof, it will be understood by those skilled in the art that various changes in form and details may be made therein without departing from the scope of the invention encompassed by the appended claims.

1. A purified persistent fetal vascular tissue cell comprising a neuronal progenitor marker.

2. The use of the cell of claim 1 for transplantation.

3. The cell of claim 1, wherein said cell comprises a human cell.

4. The cell of claim 1, wherein said neuronal progenitor marker comprises nestin, Pax6, or Ki67.

5. The cell of claim 4, wherein said cell further comprises a retinal neuronal marker.

6. The cell of claim 4, wherein said retinal neuronal marker comprises β-III-tubulin or Brn3a.

7. The cell of claim 1, wherein said cell comprises a neural morphological phenotype or express a mature neuronal marker in the presence of a neural phenotype induction factor.

8. The cell of claim 1, wherein said cell comprises a mature neuronal marker selected from the group consisting of β-III-tubulin, synaptophysin, or NF200.

9. The cell of claim 7, wherein said factor comprises retinoic acid or a neurotrophin.

10. The cell of claim 1, wherein said cell further comprises a nucleic acid encoding an exogenous neuroprotective factor or secretes said factor.

11. The cell of claim 10, wherein said cell comprises insulin-like growth factor-1 (IGF-1) or insulin-like growth factor-binding protein (IGFBP).

12. The cell of claim 1, wherein said cell comprises a nucleic acid encoding a reporter gene.

13. A cell-based method of therapy, comprising providing a purified population of human persistent fetal vasculature neural progenitor cells and transplanting said cells into an ocular tissue of a recipient subject.

14. The method of claim 13, wherein said cells are transplanted into an inner retina location of an eye.

15. The method of claim 13, wherein said subject has been diagnosed with a degenerative disease of an eye.

16. The method of claim 13, wherein said degenerative disease comprises glaucoma.

17. The method of claim 13, wherein said degenerative disease comprises optic neuropathy, optic neuritis, or a mitochondrial optic neuropathy.

18. The method of claim 13, wherein said cells have been modified to increase expression of insulin-like growth factor-1 (IGF-1) or insulin-like growth factor-binding protein (IGFBP)-1.

19. The method of claim 18, wherein said cells have been transfected with a nucleic acid encoding IGF-1, or IGFBP-1.

20. A purified population of human persistent fetal vasculature neural progenitor cells comprising an heterologous nucleic acid encoding a neuroprotective polypeptide.

21. The purified population of human persistent fetal vasculature neural progenitor cells of claim 20 wherein the heterologous nucleic acid encoding a neuroprotective polypeptide encodes IGF-1 or a neuroprotective fragment thereof.

22. A method of promoting survival or axonal outgrowth of a retinal ganglion cell, comprising contacting said cell with the population of claim 20.

23. A method of conferring neuroprotection to a retinal ganglion cell in a subject, comprising administering to an ocular tissue the cell of claim 20.

24. The method of claim 23, wherein said cell secretes IGF-1.

25. The method of claim 23, wherein said subject is suffering from or at risk of developing a neurodegenerative disease of an eye.

26. The use of the cell of claim 1 for drug discovery, wherein said cell comprises nucleic acid encoding a reporter gene.


SEQUENCE LISTING (25 sequences)

SEQ ID NO: 1 - DNA, 21 bp, Artificial Sequence
  Synthesized primer
aggtattacgagactggctcc

SEQ ID NO: 2 - DNA, 21 bp, Artificial Sequence
  Synthesized primer
gaaacagccatagagggcaaa

SEQ ID NO: 3 - DNA, 19 bp, Artificial Sequence
  Synthesized primer
actgccttcgaccgcttac

SEQ ID NO: 4 - DNA, 22 bp, Artificial Sequence
  Synthesized primer
ctccttccagacgatgaaaaca

SEQ ID NO: 5 - DNA, 21 bp, Artificial Sequence
  Synthesized primer
gcttccccatcaacttcctca

SEQ ID NO: 6 - DNA, 20 bp, Artificial Sequence
  Synthesized primer
atgcccgagaaaatgaaatg

SEQ ID NO: 7 - DNA, 20 bp, Artificial Sequence
  Synthesized primer
cgcctcatcattttcatcct

SEQ ID NO: 8 - DNA, 20 bp, Artificial Sequence
  Synthesized primer
ctgcactgtctgagcgagag

SEQ ID NO: 9 - DNA, 22 bp, Artificial Sequence
  Synthesized primer
tgaaggtcctctacttatccgc

SEQ ID NO: 10 - DNA, 19 bp, Artificial Sequence
  Synthesized primer
atggggaaggtgaaggtcg

SEQ ID NO: 11 - DNA, 22 bp, Artificial Sequence
  Synthesized primer
tcccgcttatactgggctattt

SEQ ID NO: 12 - DNA, 23 bp, Artificial Sequence
  Synthesized primer
tggttttccagagtcttcagtga

SEQ ID NO: 13 - DNA, 21 bp, Artificial Sequence
  Synthesized primer
cagagccatgatgtagctcag

SEQ ID NO: 14 - DNA, 20 bp, Artificial Sequence
  Synthesized primer
gccagtgtcccctcaatgaa

SEQ ID NO: 15 - DNA, 23 bp, Artificial Sequence
  Synthesized primer
agtatccatgcagagaggtgtag

SEQ ID NO: 16 - DNA, 20 bp, Artificial Sequence
  Synthesized primer
agcatctttgttgcacgttg

SEQ ID NO: 17 - DNA, 20 bp, Artificial Sequence
  Synthesized primer
cattgttggagcctgatcct

SEQ ID NO: 18 - DNA, 20 bp, Artificial Sequence
  Synthesized primer
acctgaggtggaagatgtgg

SEQ ID NO: 19 - DNA, 19 bp, Artificial Sequence
  Synthesized primer
gcactgtgacgttctggga

SEQ ID NO: 20 - DNA, 22 bp, Artificial Sequence
  Synthesized primer
ggggtcattgatggcaacaata

SEQ ID NO: 21 - DNA, 24 bp, Artificial Sequence
  Synthesized primer
agatgcactgcagtttgtgtgtgg

SEQ ID NO: 22 - DNA, 24 bp, Artificial Sequence
  Synthesized primer
tctacaattccagtctgtggcgct

SEQ ID NO: 23 - DNA, 20 bp, Artificial Sequence
  Synthesized primer
ggcctccaaggagtaagacc

SEQ ID NO: 24 - DNA, 20 bp, Artificial Sequence
  Synthesized primer
aggggtctacatggcaactg

SEQ ID NO: 25 - protein, 119 aa, Homo sapiens
MALCLLTFTSSATAGPETLCGAELVDALQFVCGDRGFYFNKPTGYGSSSRRAPQTGIVDECCFRSCDLRRLEMYCAPLKPAKSARSVRAQRHTDMPKTQKEVHLKNASRGSAGNKNYRM